SEQUENCE LISTING (15 sequences)

SEQ ID NO: 1 - protein, 37 aa, Homo sapiens
HDEFERHAEGTFTSDVSSYLEGQAAKEFIAWLVKGRG

SEQ ID NO: 2 - protein, 31 aa, Homo sapiens
HAEGTFTSDVSSYLEGQAAKEFIAWLVKGRG

SEQ ID NO: 3 - protein, 29 aa, Homo sapiens
HAEGTFTSDVSSYLEGQAAKEFIAWLVKG

SEQ ID NO: 4 - protein, 30 aa, Artificial Sequence
  synthesized
HXEGTFTSDVSSYLEGQAAKEFIAWLVKXR

SEQ ID NO: 5 - protein, 14 aa, Artificial Sequence
  synthesized
QAAKEFIAWLVKXR

SEQ ID NO: 6 - protein, 5 aa, Artificial Sequence
  synthesized
SYLEG

SEQ ID NO: 7 - protein, 19 aa, Artificial Sequence
  synthesized
SYLEGQAAKEFIAWLVKXR

SEQ ID NO: 8 - protein, 11 aa, Artificial Sequence
  synthesized
HXEGTFTSDVS

SEQ ID NO: 9 - protein, 30 aa, Artificial Sequence; identical to SEQ ID NO: 4
  synthesized

SEQ ID NO: 10 - protein, 13 aa, Artificial Sequence
  synthesized
QAAKEFIAWLVKX

SEQ ID NO: 11 - protein, 16 aa, Artificial Sequence
  synthesized
HXEGTFTSDVSSYLEG

SEQ ID NO: 12 - protein, 10 aa, Artificial Sequence
  synthesized
SYLEGQAAKE

SEQ ID NO: 13 - protein, 20 aa, Artificial Sequence
  synthesized
HXGTFTSDVSSYLEGQAAKE

SEQ ID NO: 14 - protein, 9 aa, Artificial Sequence
  synthesized
FIAWLVKXR

SEQ ID NO: 15 - protein, 9 aa, Artificial Sequence
  synthesized
EFIAWLVKX

(19) United States
(12) Patent Application Publication
(10) Pub. No.: US 2010/0292435 A1
(43) Pub. Date: Nov. 18, 2010

(54) INSULINOTROPIC PEPTIDE SYNTHESIS USING SOLID AND SOLUTION PHASE COMBINATION TECHNIQUES

(76) Inventors: LIN CHEN, SUPERIOR, CO (US); YEUN-KWEI HAN, LOUISVILLE, CO (US); CHRISTOPHER R. ROBERTS, BETHOUD, CO (US)

Correspondence Address: Grant D. Green;Patent Department, M/S A2-250, Roche Palo Alto LLC, 3431 Hillview Avenue, Palo Alto, CA, 94304, US

(21) Appl. No.: 12/770,907

(22) Filed: Apr. 30, 2010

Related U.S. Application Data
(60) Provisional application No. 61/174,662, filed on May 1, 2009.

(51) Int. Cl. C07K 14/00; C07K 7/08

(52) U.S. Cl. 530/324; 530/326; 530/327; 530/328

(57) ABSTRACT

The present invention relates to the preparation of insulinotropic peptides that are synthesized using a solid and solution phase (“hybrid”) approach. Generally, the approach includes synthesizing three different peptide intermediate fragments using solid phase chemistry. Solution phase chemistry is then used to couple the second fragment and the first fragment. Alternatively, a different second fragment is coupled to a first fragment in the solid phase. Then, solution phase chemistry is then used to add the third fragment, whereby the third fragment is coupled to the coupled first and second fragments in the solution phase. The present invention is very useful for forming insulinotropic peptides such as GLP-1(7-36) and its natural and non-natural counterparts.

### CROSS REFERENCE TO PRIOR APPLICATIONS

This application claims the benefit of priority to U.S. Provisional Application 61/174,662, filed May 1, 2009, which is hereby incorporated by reference in its entirety.

### REFERENCE TO SEQUENCE LISTING

This application contains a Sequence Listing submitted as an electronic text file named “R0483B_ST25.txt”, having a size in bytes of 6 kb, and created on Feb. 24, 2010. The information contained in this electronic file is hereby incorporated by reference in its entirety pursuant to 37 CFR §1.52(e)(5).

### FIELD OF THE INVENTION

The invention relates to methods for preparing insulinotropic peptides, particularly glucagon-like peptide-1 (GLP-1) and counterparts thereof, using solid- and solution-phase processes. The present invention further relates to intermediate peptide fragments that can be used in these methods.

### BACKGROUND OF THE INVENTION

Many methods for peptide synthesis are described in the literature (for example, see U.S. Pat. No. 6,015,881; Mergler et al. (1988) Tetrahedron Letters 29:4005-4008; Mergler et al. (1988) Tetrahedron Letters 29:4009-4012; Kamber et al. (eds), Peptides, Chemistry and Biology, ESCOM, Leiden (1992) 525-526; Riniker et al. (1993) Tetrahedron Letters 49:9307-9320; Lloyd-Williams et al. (1993) Tetrahedron Letters 49:11065-11133; and Andersson et al. (2000) Biopolymers 55:227-250. The various methods of synthesis are distinguished by the physical state of the phase in which the synthesis takes place, namely liquid phase or solid phase.

In solid phase peptide synthesis (SPPS), an amino acid or peptide group is bound to a solid support resin. Then, successive amino acids or peptide groups are attached to the support-bound peptide until the peptide material of interest is formed. The support-bound peptide is then typically cleaved from the support and subject to further processing and/or purification. In some cases, solid phase synthesis yields a mature peptide product; in other cases the peptide cleaved from the support (i.e., a “peptide intermediate fragment”) is used in the preparation of a larger, mature peptide product.

Peptide intermediate fragments generated from solid phase processes can be coupled together in the solid phase or in a liquid phase synthetic process (herein referred to as “solution phase synthesis”). Solution phase synthesis can be particularly useful in cases where the synthesis of a useful mature peptide by solid phase is either impossible or not practical. For example, in solid phase synthesis, longer peptides eventually may adopt an irregular conformation while still attached to the solid support, making it difficult to add additional amino acids or peptide material to the growing chain. As the peptide chain becomes longer on the support resin, the efficiency of process steps such as coupling and deprotection may be compromised. This, in turn, can result in longer processing times to compensate for these problems, in addition to incremental losses in starting materials, such as activatable amino acids, co-reagents, and solvents. These problems can increase as the length of the peptide increases.

Therefore, it is relatively uncommon to find mature peptides of greater than 30 amino acids in length synthesized in a single fragment using only a solid phase procedure. Instead, individual fragments may be separately synthesized on the solid phase, and then coupled in the solid and/or solution phase to build the desired peptide product. This approach requires careful selection of fragment candidates. While some general principles can guide fragment selection, quite often empirical testing of fragment candidates is required. Fragment strategies that work in one context may not work in others. Even when reasonable fragment candidates are uncovered, process innovations may still be needed for a synthesis strategy to work under commercially reasonable conditions. Therefore, peptide synthesis using hybrid schemes are often challenging, and in many cases it is difficult to predict what problems are inherent in a synthesis scheme until the actual synthesis is performed.

In solution phase coupling, two peptide intermediate fragments, or a peptide intermediate fragment and a reactive amino acid, are coupled in an appropriate solvent, usually in the presence of additional reagents that promote the efficiency and quality of the coupling reaction. The peptide intermediate fragments are reactively arranged so the N-terminal of one fragment becomes coupled to the C-terminal of the other fragment, or vice versa. In addition, side chain protecting groups, which are present during solid phase synthesis, are commonly retained on the fragments during solution phase coupling to ensure the specific reactivity of the terminal ends of the fragments. These side chain protecting groups are typically not removed until a mature peptide has been formed.

Modest improvements in one or more steps in the overall synthetic scheme can amount to significant improvements in the preparation of the mature peptide. Such improvements can lead to a large overall saving in time and reagents, and can also significantly improve the purity and yield of the final product.

While the discussion of the importance of improvements in hybrid synthesis is applicable to any sort of peptide produced using these procedures, it is of particular import in the context of peptides that are therapeutically useful and that are manufactured on a scale for commercial medical use. Synthesis of larger biomolecular pharmaceuticals, such as therapeutic peptides, can be very expensive. Because of the cost of reagents, synthesis time, many synthesis steps, in addition to other factors, very small improvements in the synthetic process of these larger biomolecular pharmaceuticals can have a significant impact on whether it is even economically feasible to produce such a pharmaceutical. Such improvements are necessary due to these high production costs for larger biomolecular pharmaceuticals as supported by the fact that, in many cases, there are few, if any, suitable therapeutic alternatives for these types of larger biomolecular pharmaceuticals.

This is clearly seen in the case of the glucagon-like peptide-1 (GLP-1) and its counterparts. These peptides have been implicated as possible therapeutic agents for the treatment of type 2 non-insulin-dependent diabetes mellitus as well as related metabolic disorders, such as obesity. Gutniak, M. K., et al., Diabetes Care 1994:17:1039-44.

Lopez et al. determined that native GLP-1 was 37 amino acid residues long. Lopez, L. C., et al., Proc. Natl. Acad. Sci. USA., 80:5485-5489 (1983). This determination was confirmed by the work of Uttenthal, L. O., et al., J. Clin. Endocrinal. Metabol., 61:472-479 (1985). Native GLP-1 may be represented by the notation GLP-1 (1-37). This notation indicates that the peptide has all amino acids from 1 (N-terminus) through 37 (C-terminus). Native GLP-1 (1-37) has the amino acid sequence according to SEQ ID NO. 1:
HDEFERHAEGTFTSDVSSYLEGQAAKEFIAWLVKGRG

### SUMMARY OF THE INVENTION

The present application relates to the preparation of insulinotropic peptides that are synthesized using a solid and solution phase (“hybrid”) approach. In one method, the approach includes synthesizing three different peptide intermediate fragments using solid phase chemistry. Solution phase chemistry is then used to add additional amino acid material to one of the fragments. The fragments are then coupled together in the solution phase. The present invention is very useful for forming insulinotropic peptides such as GLP-1, GLP-1 (7-36) and natural and non-natural counterparts of these, particularly GLP-1 (7-36) and its natural and non-natural counterparts.

The application provides a method of making an insulinotropic peptide, comprising the steps of:
a) providing a first peptide fragment including the amino acid sequence of (SEQ ID NO. 5)

Z-QAAKEFIAWLVKX³⁵R-NH₂

wherein
Z is H—;
X³⁵ is an achiral, optionally sterically hindered amino acid residue; and
one or more residues of the sequence optionally includes side chain protection;
b) providing a second peptide fragment including the amino acid sequence of (SEQ ID NO. 6)

Z-SYLEG

wherein
Z is an N-terminal protecting group; and
one or more residues of the sequence optionally includes side chain protection;
c) coupling the first peptide fragment to the second peptide fragment in solution in order to provide a third peptide fragment including the amino acid sequence of (SEQ ID NO. 7)

Z-SYLEGQAAKEFIAWLVKX³⁵R-NH₂

wherein
Z is an N-terminal protecting group;
X³⁵ is an achiral, optionally sterically hindered amino acid residue; and
one or more residues of the sequence optionally includes side chain protection;
d) removing the N-terminal protecting group of the third peptide fragment to afford a fourth peptide fragment including the amino acid sequence of (SEQ ID NO. 7)

Z-SYLEGQAAKEFIAWLVKX³⁵R-NH₂

wherein
Z is H—;
X³⁵ is an achiral, optionally sterically hindered amino acid residue; and one or more residues of the sequence optionally includes side chain protection;
e) providing a fifth peptide fragment including the amino acid sequence of (SEQ ID NO. 8)

Z-HX⁸EGTFTSDVS-B′

wherein
X⁸ is an achiral, optionally sterically hindered amino acid residues;
Z is an N-terminal protecting group;
B′ is —OH; and
one or more residues of the sequence optionally includes side chain protection;
f) coupling the fifth peptide fragment to the fourth peptide fragment in solution to provide an insulinotropic peptide including the amino acid sequence of (SEQ ID NO. 9)

Z-HX⁸EGTFTSDVSSYLEGQAAKEFIAWLVKX³⁵R-NH₂

wherein
Z is an N-terminal protecting group;
X⁸ and X³⁵ are each independently achiral, optionally sterically hindered amino acid residues; and
one or more residues of the sequence optionally includes side chain protection.

The application provides the above method, further comprising the steps of:
g) removing the N-terminal protecting group of the insulinotropic peptide resulting from step f) to afford the insulinotropic peptide including amino acid sequence of (SEQ ID NO. 9)

Z-HX⁸EX¹⁰TFTSDVSSYLEGQAAKEFIAWLVKX³⁵R-NH₂

wherein
Z is H—;
X⁸ and X³⁵ are each independently achiral, optionally sterically hindered amino acid residues; and
one or more residues of the sequence optionally includes side chain protection; and
h) contacting the insulinotropic peptide resulting from step g) with acid in order to deprotect the amino acid side chains to afford the deprotected insulinotropic peptide including amino acid sequence of (SEQ ID NO. 9)

Z-HX⁸EGTFTSDVSSYLEGQAAKEFIAWLVKX³⁵R-NH₂

wherein
Z is H—; and
X⁸ and X³⁵ are each independently achiral, optionally sterically hindered amino acid residues.

The application provides the above method, wherein the deprotected insulinotropic peptide resulting from step h) has the amino acid sequence (SEQ. ID No. 4)

HAibEGTFTSDVSSYLEGQAAKEFIAWLVKAibR

The application provides a method of making an insulinotropic peptide, comprising the steps of:
a) providing a first peptide fragment including the amino acid sequence of (SEQ ID NO. 8)

Z-HX⁸EGTFTSDVS-B′

wherein
X⁸ is an achiral, optionally sterically hindered amino acid residues;
Z is an N-terminal protecting group;
B′ is —OH; and
one or more residues of the sequence optionally includes side chain protection;
b) providing a second peptide fragment including the amino acid sequence of (SEQ ID NO. 6)

Z-SYLEG-B′

wherein
B′ is a solid phase resin;
Z is H—; and
one or more residues of the sequence optionally includes side chain protection;
c) coupling the first peptide fragment to the second peptide fragment in order to provide a third peptide fragment including the amino acid sequence of (SEQ ID NO. 11)

Z-HX⁸EGTFTSDVSSYLEG-B′

wherein
B′ is a solid phase resin;
Z is an N-terminal protecting group; and
one or more residues of the sequence optionally includes side chain protection;
d) removing the third peptide fragment from the solid phase resin to provide a fourth peptide fragment including the amino acid sequence of (SEQ ID NO. 11)

Z-HX⁸EGTFTSDVSSYLEG-B′

wherein
B′ is —OH;
Z is an N-terminal protecting group; and
one or more residues of the sequence optionally includes side chain protection;
e) providing a fifth peptide fragment including the amino acid sequence of (SEQ ID NO. 5)

Z-QAAKEFIAWLVKX³⁵R-NH₂

wherein
Z is H—;
X³⁵ is an achiral, optionally sterically hindered amino acid residue; and
one or more residues of the sequence optionally includes side chain protection;
f) coupling the fourth peptide fragment to the fifth peptide fragment in solution to provide an insulinotropic peptide including the amino acid sequence of (SEQ ID NO. 9)

Z-HX⁸EGTFTSDVSSYLEGQAAKEFIAWLVKX³⁵R-NH₂

wherein
Z is an N-terminal protecting group;
X⁸ and X³⁵ are each independently achiral, optionally sterically hindered amino acid residues; and
one or more residues of the sequence optionally includes side chain protection.

The application provides the above method, further comprising the steps of:
g) removing the N-terminal protecting group of the insulinotropic peptide resulting from step f) to afford an insulinotropic peptide including the amino acid sequence of (SEQ ID NO. 9)

Z-HX⁸EGTFTSDVSSYLEGQAAKEFIAWLVKX³⁵R-NH₂

wherein
Z is H—;
X⁸ and X³⁵ are each independently achiral, optionally sterically hindered amino acid residues; and
one or more residues of the sequence optionally includes side chain protection;

h) contacting the insulinotropic peptide resulting from step g) with acid in order to deprotect the amino acid side chains to afford the deprotected insulinotropic peptide including amino acid sequence of (SEQ ID NO. 9)

Z-HX⁸EGTFTSDVSSYLEGQAAKEFIAWLVKX³⁵R-NH₂

wherein
Z is H—; and
X⁸ and X³⁵ are each independently achiral, optionally sterically hindered amino acid residues.

The application provides the above method, wherein the deprotected insulinotropic peptide has the amino acid sequence (SEQ. ID No. 4)

HAibEGTFTSDVSSYLEGQAAKEFIAWLVKAibR-NH₂

The application provides a method of making an insulinotropic peptide, comprising the steps of:
a) providing a first peptide fragment including the amino acid sequence of (SEQ ID NO. 12)

Z-SYLEGQAAKE-B′

wherein
Z is H—; and
B′ is a solid phase resin;
b) providing a second peptide fragment including the amino acid sequence of (SEQ ID NO. 8)

Z-HX⁸EGTFTSDVS-B′

wherein
X⁸ is an achiral, optionally sterically hindered amino acid residues;
Z is an N-terminal protecting group;
B′ is —OH; and
one or more residues of the sequence optionally includes side chain protection;
c) coupling the second peptide fragment to the first peptide fragment to provide a third peptide fragment including the amino acid sequence of (SEQ ID NO. 13)

Z-HX⁸EGTFTSDVSSYLEGQAAKE-B′

wherein
Z is an N-terminal protecting group;
B′ is a solid phase resin;
X⁸ is an achiral, optionally sterically hindered amino acid residues; and
one or more residues of the sequence optionally includes side chain protection.

The application provides the above method, further comprising the steps of:
d) removing the third peptide fragment from the solid phase resin to provide a fourth peptide fragment including amino acid sequence of (SEQ ID NO. 13)

Z-HX⁸EGTFTSDVSSYLEGQAAKE-B′

wherein
Z is H—;
B′ is —OH;
X⁸ is an achiral, optionally sterically hindered amino acid residues; and
one or more residues of the sequence optionally includes side chain protection; and
e) providing a fifth peptide fragment including the amino acid sequence of (SEQ ID NO. 14)

Z-FIAWLVKX³⁵R-NH₂

wherein
X³⁵ is an achiral, optionally sterically hindered amino acid residue; and one or more residues of the sequence optionally includes side chain protection;
f) coupling the fourth peptide fragment to the fifth peptide fragment in solution to provide an insulinotropic peptide including the amino acid sequence of (SEQ ID NO. 9)

Z-HX⁸EGTFTSDVSSYLEGQAAKEFIAWLVKX³⁵R-NH₂

wherein
Z is an N-terminal protecting group;
X⁸ and X³⁵ are each independently achiral, optionally sterically hindered amino acid residues; and
one or more residues of the sequence optionally includes side chain protection;
g) removing the N-terminal protecting group of the insulinotropic peptide resulting from step f) to afford the insulinotropic peptide including amino acid sequence of (SEQ ID NO. 9)

Z-HX⁸EGTFTSDVSSYLEGQAAKEFIAWLVKX³⁵R-NH₂

wherein
Z is H—;
X⁸ and X³⁵ are each independently achiral, optionally sterically hindered amino acid residues; and
one or more residues of the sequence optionally includes side chain protection; and
h) contacting the insulinotropic peptide resulting from step g) with acid in order to deprotect the amino acid side chains to afford the deprotected insulinotropic peptide including amino acid sequence of (SEQ ID NO. 9)

Z-HX⁸EGTFTSDVSSYLEGQAAKEFIAWLVKX³⁵R-NH₂

wherein
Z is H—; and
X⁸ and X³⁵ are each independently achiral, optionally sterically hindered amino acid residues.

The application provides the above method, wherein the deprotected insulinotropic peptide has the amino acid sequence (SEQ. ID No. 4)

HAibEGTFTSDVSSYLEGQAAKEFIAWLVKAibR-NH₂

The application provides a method of making an insulinotropic peptide, comprising the steps of:
a) providing a first peptide fragment including the amino acid sequence of (SEQ ID NO. 14)

Z-FIAWLVKX³⁵R-NH₂

wherein
Z is H—;
X³⁵ is an achiral, optionally sterically hindered amino acid residue; and
one or more residues of the sequence optionally includes side chain protection;
b) providing a second peptide fragment including the amino acid sequence of (SEQ ID NO. 12)

Z-SYLEGQAAKE-B′

wherein
Z is an N-terminal protecting group;
B′ is —OH; and
one or more residues of the sequence optionally includes side chain protection;
c) coupling the first peptide fragment to the second peptide fragment in solution to provide a third peptide fragment including the amino acid sequence of (SEQ. ID NO. 7)

Z-SYLEGQAAKEFIAWLVKX³⁵R-NH₂

wherein
Z is an N-terminal protecting group;
X³⁵ is an achiral, optionally sterically hindered amino acid residues; and
one or more residues of the sequence optionally includes side chain protection;

The application provides the above method, further comprising the steps of:
d) removing the N-terminal protecting group of the third peptide fragment to afford a fourth peptide fragment including the amino acid sequence of (SEQ. ID NO. 7)

Z-SYLEGQAAKEFIAWLVKX³⁵R-NH₂

wherein
Z is H—;
X³⁵ is an achiral, optionally sterically hindered amino acid residues; and
one or more residues of the sequence optionally includes side chain protection;
e) providing a fifth peptide fragment including the amino acid sequence of (SEQ ID NO. 8)

Z-HX⁸EGTFTSDVS-B′

wherein
X⁸ is an achiral, optionally sterically hindered amino acid residues;
Z is an N-terminal protecting group;
B′ is —OH; and
one or more residues of the sequence optionally includes side chain protection;
f) coupling the fifth peptide fragment to the fourth peptide fragment in solution to provide an insulinotropic peptide including the amino acid sequence of (SEQ ID NO. 9)

Z-HX⁸EGTFTSDVSSYLEGQAAKEFIAWLVKX³⁵R-NH₂

wherein
Z is is an N-terminal protecting group; and
X⁸ and X³⁵ are each independently achiral, optionally sterically hindered amino acid residues;
g) removing the N-terminal protecting group of the insulinotropic peptide resulting from step f) to afford the insulinotropic peptide including amino acid sequence of (SEQ ID NO. 9)

Z-HX⁸EGTFTSDVSSYLEGQAAKEFIAWLVKX³⁵R-NH₂

wherein
Z is H—;
X⁸ and X³⁵ are each independently achiral, optionally sterically hindered amino acid residues; and
one or more residues of the sequence optionally includes side chain protection; and
h) contacting the insulinotropic peptide resulting from step h) with acid in order to deprotect the amino acid side chains to afford the deprotected insulinotropic peptide including amino acid sequence of (SEQ ID NO. 9)

Z-HX⁸EGTFTSDVSSYLEGQAAKEFIAWLVKX³⁵R-NH₂

wherein
Z is H—; and
X⁸ and X³⁵ are each independently achiral, optionally sterically hindered amino acid residues.

The application provides the above method, wherein the deprotected insulinotropic peptide has the amino acid sequence (SEQ. ID No. 4)

HAibEGTFTSDVSSYLEGQAAKEFIAWLVKAibR-NH₂

The application provides a peptide of the amino acid sequence (SEQ ID NO. 5)

Z-QAAKEFIAWLVKX³⁵R-NH₂

wherein
Z is H— or an N-terminal protecting group;
X³⁵ is an achiral, optionally sterically hindered amino acid residue; and
one or more residues of the sequence optionally includes side chain protection.

The application provides a peptide of the amino acid sequence (SEQ ID NO. 7)

Z-SYLEGQAAKEFIAWLVKX³⁵R-NH₂

wherein
Z is H— or an N-terminal protecting group;
X³⁵ is an achiral, optionally sterically hindered amino acid residue; and
one or more residues of the sequence optionally includes side chain protection.

The application provides a peptide of the amino acid sequence (SEQ ID NO. 8)

Z-HX⁸EGTFTSDVS-B′

wherein
X⁸ is an achiral, optionally sterically hindered amino acid residues;
Z is H— or an N-terminal protecting group;
B′ is —OH or a solid phase resin; and
one or more residues of the sequence optionally includes side chain protection.

The application provides a peptide of the amino acid sequence (SEQ ID NO. 11)

Z-HX⁸EGTFTSDVSSYLEG-B′

wherein
B′ is —OH or a solid phase resin;
Z is H— or an N-terminal protecting group; and
one or more residues of the sequence optionally includes side chain protection.

The application provides a peptide of the amino acid sequence (SEQ ID NO. 12)

Z-SYLEGQAAKE-B′

wherein
Z is H— or an N-terminal protecting group; and
B′ is —OH or a solid phase resin.

The application provides a peptide of the amino acid sequence (SEQ ID NO. 13)

Z-HX⁸EGTFTSDVSSYLEGQAAKE-B′

wherein
Z is H— or an N-terminal protecting group;
B′ is —OH or a solid phase resin;
X⁸ is an achiral, optionally sterically hindered amino acid residues; and
one or more residues of the sequence optionally includes side chain protection.

The application provides a peptide of the amino acid sequence (SEQ. ID NO. 7)

Z-SYLEGQAAKEFIAWLVKX³⁵R-NH₂

wherein
Z is H— or an N-terminal protecting group;
X³⁵ is an achiral, optionally sterically hindered amino acid residues; and
one or more residues of the sequence optionally includes side chain protection.

The application provides a peptide of the amino acid sequence (SEQ. ID NO. 14)

Z-FIAWLVKX³⁵R-NH₂

wherein
Z is H— or an N-terminal protecting group;
X³⁵ is an achiral, optionally sterically hindered amino acid residues; and
one or more residues of the sequence optionally includes side chain protection.

The application further provides any of the above peptides, wherein Z is Fmoc.

In one aspect, any of the above methods may employ N-terminus histidine protecting groups (N-terminal protecting groups) selected from the group consisting of Fmoc (9-fluorenylmethoxycarbonyl), Boc (t-butyloxycarbonyl), CBz (benzyloxycarbonyl or Z), Dts (dithiasuccinoyl), Rdtc (R=Alkyl or Aryl, dtc=dithiocarbamate), DBFmoc (2,7-di-t-butylFmoc or 1,7-di-t-butylfluoren-9-ylmethoxycarbonyl), Alloc (allyloxycarbonyl), pNZ (p-nitrobenzyloxycarbonyl), Nsc ([[2-[(4-nitrophenyl)sulfonyl]ethoxy]carbonyl]), Msc (2-methylsulfonylethoxycarbonyl), MBz (4-methoxyCBz), Bpoc [(1-[1,1′-biphenyl]-4-yl-1-methylethoxy)carbonyl], Bnpeoc [[2,2-bis(4-nitrophenyl)ethoxy]carbonyl], CBz [(phenylmethoxy)carbonyl], Aoc [(1,1-dimethylpropoxy)carbonyl], and Moz [[(4-methoxyphenyl)methoxy]carbonyl], wherein if the N-terminus histidine protecting group may be removed in the global side-chain deprotection step using acid, prior removal of the N-terminus histidine protecting group is not required.

### DETAILED DESCRIPTION OF THE INVENTION

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

The present invention is directed to synthetic methods for making peptides such as the glucagon-like peptide-1 (GLP-1), and natural and non-natural insulinotropically active counterparts thereof, using solid and/or solution phase techniques. Peptide molecules of the invention may be protected, unprotected, or partially protected. Protection may include N-terminus protection, side chain protection, and/or C-terminus protection. While the invention is generally directed at the synthesis of these glucagon-like peptides, their counterparts, fragments and their counterparts, and fusion products and their counterparts of these, the inventive teachings herein can also be applicable to the synthesis of other peptides, particularly those that are synthesized using a combination of solid phase and solution phase approaches. The invention is also applicable to the synthesis of peptide intermediate fragments associated with impurities, particularly pyroglutamate impurities. Preferred GLP-1 molecules useful in the practice of the present invention include natural and non-natural GLP-1 (7-36) and counterparts thereof.

As used herein, the term “including the amino acid sequence” preferably means “having the amino acid sequence”.

As used herein, a “counterpart” refers to natural and non-natural analogs, derivatives, fusion compounds, salts, or the like of a peptide. As used herein, a peptide analog generally refers to a peptide having a modified amino acid sequence such as by one or more amino acid substitutions, deletions, inversions, and/or additions relative to another peptide or peptide counterpart. Substitutions may involve one or more natural or non-natural amino acids. Substitutions preferably may be conservative or highly conservative. A conservative substitution refers to the substitution of an amino acid with another that has generally the same net electronic charge and generally the same size and shape. For instance, amino acids with aliphatic or substituted aliphatic amino acid side chains have approximately the same size when the total number of carbon and heteroatoms in their side chains differs by no more than about four. They have approximately the same shape when the number of branches in their side chains differs by no more than about one or two. Amino acids with phenyl or substituted phenyl groups in their side chains are considered to have about the same size and shape. Listed below are five groups of amino acids. Replacing an amino acid in a compound with another amino acid from the same groups generally results in a conservative substitution.

Group I: glycine, alanine, valine, leucine, isoleucine, serine, threonine, cysteine, methionine and non-naturally occurring amino acids with C₁-C₄ aliphatic or C₁-C₄ hydroxyl substituted aliphatic side chains (straight chained or monobranched).

Group II: glutamic acid, aspartic acid and nonnaturally occurring amino acids with carboxylic acid substituted C₁-C₄ aliphatic side chains (unbranched or one branch point).

Group III: lysine, ornithine, arginine and nonnaturally occurring amino acids with amine or guanidino substituted C₁-C₄ aliphatic side chains (unbranched or one branch point).

Group IV: glutamine, asparagine and non-naturally occurring amino acids with amide substituted C₁-C₄ aliphatic side chains (unbranched or one branch point). Group V: phenylalanine, phenylglycine, tyrosine and tryptophan.

As used herein, the term “counterpart” more preferably refers to the salts of a peptide, or to the derivatives thereof that are amidated at the C-terminus.

A “highly conservative substitution” is the replacement of an amino acid with another amino acid that has the same functional group in the side chain and nearly the same size and shape. Amino acids with aliphatic or substituted aliphatic amino acid side chains have nearly the same size when the total number carbon and heteroatoms in their side chains differs by no more than two. They have nearly the same shape when they have the same number of branches in their side chains. Examples of highly conservative substitutions include valine for leucine, threonine for serine, aspartic acid for glutamic acid and phenylglycine for phenylalanine

A “peptide derivative” generally refers to a peptide, a peptide analog, or other peptide counterpart having chemical modification of one or more of its side groups, alpha carbon atoms, terminal amino group, and/or terminal carboxyl acid group. By way of example, a chemical modification includes, but is not limited to, adding chemical moieties, creating new bonds, and/or removing chemical moieties. Modifications at amino acid side groups include, without limitation, acylation of lysine e-amino groups, N-alkylation of arginine, histidine, or lysine, alkylation of glutamic or aspartic carboxylic acid groups, and deamidation of glutamine or asparagine. Modifications of the terminal amino group include, without limitation, the des-amino, N-lower alkyl, N-di-lower alkyl, and N-acyl (e.g., —CO-lower alkyl) modifications. Modifications of the terminal carboxy group include, without limitation, the amide, lower alkyl amide, dialkyl amide, and lower alkyl ester modifications. Thus, partially or wholly protected peptides constitute peptide derivatives.

In the practice of the present invention, a compound has “insulinotropic” activity if it is able to stimulate, or cause the stimulation of, or help cause the stimulation of the synthesis or expression of the hormone insulin. In preferred modes of practice, insulinotropic activity can be demonstrated according to assays described in U.S. Pat. Nos. 6,887,849 and 6,703,365.

In preferred embodiments, the present invention provides methodologies for synthesizing synthetic (X⁸, X³⁵)GLP-1(7-36) peptides having the following formula (SEQ. ID NO. 9):

HX⁸EGTFTSDVSSYLEGQAAKEFIAWLVKX³⁵R-NH₂

and counterparts thereof, wherein each of the symbols X at positions, 8 and 35 independently denotes an achiral, optionally sterically hindered amino acid residue. Either of the X⁸ and/or X³⁵ residues optionally may include side chain protecting group(s). Peptides according to this formula differ from the native GLP-1(7-36) at least in that the achiral, optionally sterically hindered X⁸ and X³⁵ residues are substituted for the native amino acid residues at positions 8 and 35. The use of the achiral X⁸ and X³⁵ amino acids not only help to stabilize the resultant peptide, but it has also now been discovered that the use of these amino acids as linker of building blocks also facilitate the synthesis route of the present invention as shown in Scheme 1 and described further below.

A particularly preferred embodiment of a (X⁸, X³⁵)GLP-11 (7-36) peptide that may be synthesized in accordance with principles of the present invention includes a peptide according to the formula (SEQ. ID NO. 4):

HAibEGTFTSDVSSYLEGQAAKEFIAWLVKAibR-NH₂

and counterparts thereof, which preferably (as shown) is amidated at the C-terminus. This peptide uses the achiral residue of alpha-aminoisobutyric acid (shown schematically by the abbreviation Aib) as both X⁸ and X³⁵, preferably has an amide at the C-terminus, uses a residue of the native G at the 10 position, and may be designated by the formula (Aib^{8,35}) GLP-1 (7-36)-NH₂. This notation indicates that an amino acid residue corresponding to the amino acid “Aib” appears at the 8 and 35 positions in place of the native alanine. The achiral alpha-aminoisobutric acid, also is known as methylalanine The peptide according to SEQ. ID NO. 4 is described in EP 1137667 B1. The presence of the Aib residues at the 8 and 35 positions slows metabolic degradation in the body, making this peptide much more stable in the body than the native GLP-1 (7-36) peptide.

The present invention provides improved methodologies for making GLP-1(7-36) peptides such as the (Aib^{8,35})GLP-1(7-36)-NH₂. By way of example, Scheme 1 and Scheme 2 show illustrative schemes for synthesizing GLP-1(7-36) peptides and their counterparts. Scheme 1 and Scheme 2 are believed to be particularly suitable for the scaled-up synthesis of GLP-1(7-36) peptides. Scaled-up procedures are typically performed to provide an amount of peptide useful for commercial distribution. For example the amount of peptide in a scaled-up procedure can be 500 g, or 1 kg per batch, and more typically tens of kg to hundreds of kg per batch or more. In preferred embodiments, the inventive methods can provide such improvements as reduction in processing (synthesis) time, improvements in the yield of products, improvements in product purity, and/or reduction in amount of reagents and starting materials required.

The synthesis shown in Scheme 1 uses a combination of solid and solution phase techniques to prepare the peptide product.

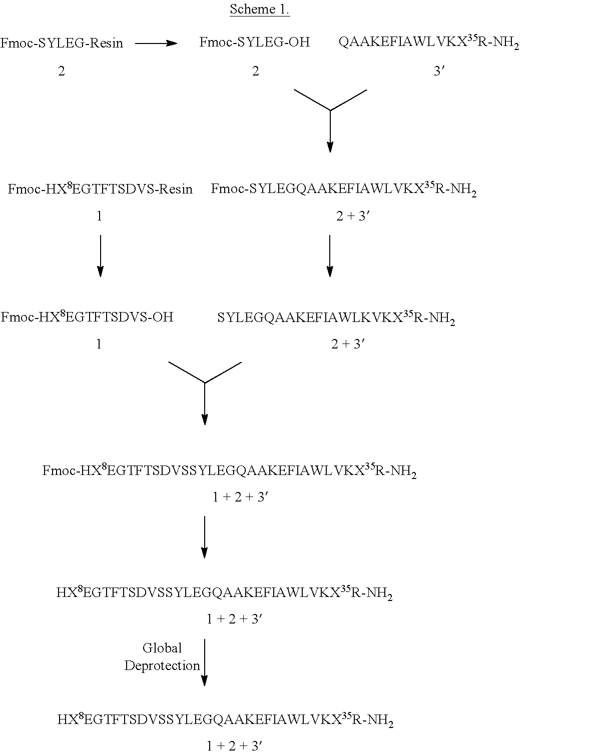

As shown, Scheme 1 involves synthesizing peptide intermediate fragments 1, 2 and 3 on the solid phase. Fragment 1 is a peptide fragment including amino acid residues according to SEQ ID NO. 8:

HX⁸EGTFTSDVS

wherein X⁸ is as defined above, or is a counterpart thereof including the X⁸ residues. One or more of the amino acid residues may include side chain protecting groups in accordance with conventional practices. In some embodiments, the peptide fragment 1 may be resin bound via the C-terminus. This fragment optionally may bear N-terminus and/or C-terminus protection groups. Fmoc has been found to be a particularly useful N-terminus histidine protecting group with respect to solid phase synthesis and solution or solid phase coupling of the peptide fragment. Trt (trityl) has also been found to be a particularly useful N-terminus histidine protecting group with respect to solid phase synthesis and solution or solid phase coupling of the peptide fragment. Boc, CBz, DTS, Rdtc (R=Alkyl or Aryl), DBFmoc (2,7-di-t-butylFmoc), Alloc, pNZ (p-nitrobenzyl ester), Nsc ([[2-[(4-nitrophenyl)sulfonyl]ethoxy]carbonyl]-), Msc (2-methylsulfonylethoxycarbonyl), and MBz (4-methoxyCBz) are also particularly useful N-terminus histidine protecting groups with respect to solid phase synthesis and solution or solid phase coupling of the peptide fragment. [(1-[1,1′-biphenyl]-4-yl-1-methylethoxy)carbonyl], [[2,2-bis(4-nitrophenyl)ethoxy]carbonyl], [(phenylmethoxy)carbonyl], [(1,1-dimethylpropoxy)carbonyl], [[(4-methoxyphenyl)methoxy]carbonyl] are particularly useful N-terminus histidine protecting groups with respect to solid phase synthesis and solution or solid phase coupling of the peptide fragment.

Fragment 1 includes the 11 amino acid residues corresponding to the amino acids in the 7 through 17 positions of the native GLP-1(7-36) peptide, and therefore may be represented by the notation (X⁸)GLP-1(7-17). In preferred embodiments, X⁸ is Aib or is a counterpart thereof including the Aib residue at the 10 position. The peptide fragment according to SEQ ID NO. 8 may be represented by the notation (Aib⁸)GLP-1(17-10) to note the substitution of Aib for the native alanine at the 8 position of the native GLP-1(7-10).

Solid phase synthesis is generally carried out in a direction from the C-terminus to the N-terminus of the fragment 1. Thus, the S¹⁷ amino acid, which is present on the C-terminal portion of the fragment, is the first amino acid residue that is coupled to the solid phase resin support. Solid phase synthesis then proceeds by consecutively adding amino acid residues in a manner corresponding to the desired sequence. The synthesis of the peptide intermediate fragment is complete after the N-terminal residue (for example, the N-terminal histidine residue (H) has been added to the nascent peptide chain.

Fragment 2 is a peptide fragment including amino acid residues according to SEQ ID NO. 6:

SYLEG

Fragment 2 includes amino acid residues generally corresponding to the amino acid residues in the 18 through 22 positions of the native GLP-1(7-36) peptide.

One or more of the amino acid residues of fragment 2 may include side chain protecting groups in accordance with conventional practices. In some embodiments, the peptide fragment 2 may be resin bound via the C-terminus. This fragment optionally may bear N-terminus and/or C-terminus protection groups. Fmoc has been found to be a particularly useful N-terminus protecting group with respect to solid phase synthesis of the peptide fragment. The peptide fragment according to SEQ ID NO. 6 may be referred to by the notation GLP-1 (18-22).

Solid phase synthesis is generally carried out in a direction from the C-terminus to the N-terminus of the fragment 1. Thus, the G amino acid, which is present on the C-terminal portion of the fragment, is the first amino acid residue that is coupled to the solid phase resin support. Solid phase synthesis then proceeds by consecutively adding amino acid residues in a manner corresponding to the desired sequence. The synthesis of the peptide intermediate fragment is complete after the N-terminal residue (for example, the N-terminal serine residue (S) has been added to the nascent peptide chain).

Fragment 3′ is a peptide fragment, or counterpart thereof, including amino acid residues according to SEQ ID NO. 5 wherein X³⁵ is as defined above, or is a

QAAKEFIAWLVKX³⁵R

counterpart thereof including the X³⁵ residue. One or more of the amino acid residues may include side chain protecting groups in accordance with conventional practices. Fragment 3′ includes the amino acid residues corresponding to the amino acids in the 23 through 36 positions of the native GLP-1(7-36) peptide, except that X³⁵ is at the 35 position in place of the native amino acid at that position. Fragment 3′ may be represented by the notation (X³⁵)GLP-1(23-36).

QAAKEFIAWLVKX³⁵

Fragment 3′ is conveniently prepared from fragment 3 (SEQ ID NO. 10). Fragment 3 is prepared by solid phase synthesis from Fmoc-Aib³⁵-O-2CT using standard coupling protocols. The lysine and tryptophan side chains were protected with Boc. The glutamic acid side chain was protected as a tent-Bu ester and the glutamine side chain was protected by a trityl group. Fragment 3 was cleaved from the resin and coupled with H-Arg (2HCl)-NH₂. Fragment 3 includes the amino acid residues corresponding to amino acids in positions 23 through 35 of native GLP-1(7-36) except that X³⁵ is Aib.

In some embodiments, the peptide fragment 3 may be resin bound via the C-terminus. This fragment optionally may bear side chain, N-terminus and/or C-terminus protection groups. Fmoc has been found to be a particularly useful N-terminus protecting group with respect to solid phase synthesis of the peptide fragment. In preferred embodiments, X³⁵ is Aib or a counterpart thereof including the Aib at the 35 position and may be represented by the notation (Aib³⁵) GLP-1(23-35) to note the substitution of Aib for the native amino acid at the 35 position of the native GLP-1(7-35).

Due to steric hindrance proximal to the X³⁵ -loaded support resin, the coupling of lysine (34) and valine (33) onto the peptide chain can be problematic. Even with an excess of amino acid, it is difficult to force these coupling reactions to completion. Solvent choice and/or end-capping can help to alleviate this problem. It has been found that the nature of the coupling solvent can impact the degree to which the coupling goes to completion. In one set of experiments, for example, coupling reactions were carried out in a 3:1 NMP/DCM, 1:1 NMP/DCM, 1:1 DMF/DCM, and 3:1 DMF/DCM. The ratios in these solvent combinations are on a volume basis. NMP refers to N-methyl pyrrolidone, DCM refers to dichloromethane, and DMF refers to dimethylformamide. It was found that the coupling reactions proceeded farther to completion when using 1:1 DMF/DCM.

End-capping after each of the lysine and valine couplings can also be used to prevent unreacted resin-supported material from proceeding in further coupling reactions. The end-capped material is more easily removed during purification if desired. Conventional end-capping techniques may be used.

Continuing to refer to Scheme 1, fragments 1, 2, and 3′ are assembled to complete the desired peptide.

Scheme 1 shows that fragment 2 is added to fragment 3′ produce a larger, intermediate fragment incorporating amino acid residues according to SEQ ID NO. 7

SYLEGQAAKEFIAWLVKX³⁵R-NH₂

wherein X³⁵ is as defined above and is preferably Aib as defined above. The intermediate fragment may be designated by the notation (X³⁵) GLP-1(18-36). To the extent that the amino acids bear side chain protection, this protection desirably is maintained through this step.

Scheme 1 further shows that fragment 1 is then added to this intermediate fragment in solution to produce the desired peptide (SEQ ID NO. 9):

HX⁸EGTFTSDVSSYLEGQAAKEFIAWLVKX³⁵R-NH₂

In alternative preferred embodiments, the present invention provides methodologies for synthesizing synthetic (X⁸, X³⁵)GLP-1 (7-36) peptides having the following formula (SEQ. ID NO. 9):

HX⁸EX¹⁰TFTSDVSSYLEGQAAKEFIAWLVKX³⁵R-NH₂

and counterparts thereof, wherein each of the symbols X at positions, 8 and 35 independently denotes an achiral, optionally sterically hindered amino acid residue. Either of the X⁸ and/or X³⁵ residues optionally may include side chain protecting group(s). Peptides according to this formula differ from the native GLP-1(7-36) at least in that the achiral, optionally sterically hindered X⁸ and X³⁵ residues are substituted for the native amino acid residues at positions 8 and 35. The use of the achiral X⁸ and X³⁵ amino acids not only help to stabilize the resultant peptide, but it has also now been discovered that the use of these amino acids as building blocks also facilitate the facile synthesis route of the present invention as shown in Scheme 1 and described further below.

A particularly preferred embodiment of a (X⁸, X³⁵) GLP-11 (7-36) peptide that may be synthesized in accordance with principles of the present invention includes a peptide according to the formula (SEQ. ID NO. 4):

HAibEGTFTSDVSSYLEGQAAKEFIAWLVKAibR-NH₂

and counterparts thereof, which preferably (as shown) is amidated at the C-terminus. This peptide uses the achiral residue of alpha-aminoisobutyric acid (shown schematically by the abbreviation Aib) as both X⁸ and X³⁵, preferably has an amide at the C-terminus, and may be designated by the formula (Aib^{8,35})GLP-1(7-36)-NH₂. This notation indicates that an amino acid residue corresponding to the amino acid “Aib” appears at the 8 and 35 positions in place of the native alanine. The achiral alpha-aminoisobutric acid, also is known as methylalanine The peptide according to SEQ ID NO. 4 is described in EP 1137667 B1. The presence of the Aib residues at the 8 and 35 positions slows metabolic degradation in the body, making this peptide much more stable in the body than the native GLP-1(7-36) peptide.

The synthesis shown in Scheme 2 uses a combination of solid and solution phase techniques to prepare the peptide product.

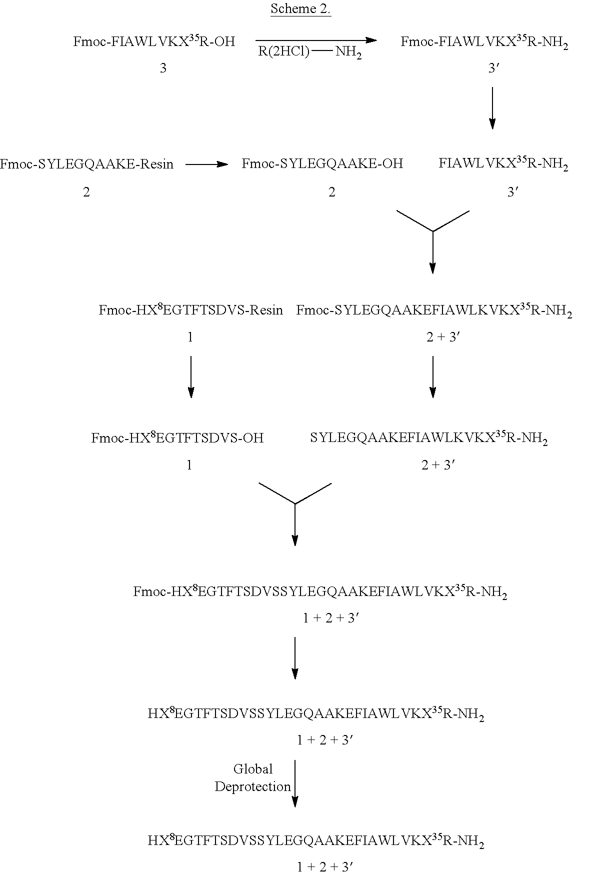

As shown, Scheme 2 involves synthesizing peptide intermediate fragments 1 and 2 on the solid phase. Fragment 1 is a peptide fragment including amino acid residues according to SEQ ID NO. 8:

HX⁸EGTFTSDVS

wherein X⁸ is as defined above, or is a counterpart thereof including the X⁸ residue. One or more of the amino acid residues may include side chain protecting groups in accordance with conventional practices. In some embodiments, the peptide fragment 1 may be resin bound via the C-terminus. This fragment optionally may bear N-terminus and/or C-terminus protection groups. Fmoc is a useful N-terminus histidine protecting group with respect to solid phase synthesis and solution or solid phase coupling of the peptide fragment. Trt (trityl) is a useful N-terminus histidine protecting group with respect to solid phase synthesis and solution or solid phase coupling of the peptide fragment. Boc (t-butyloxycarbonyl), CBz (benzyloxycarbonyl or Z), Dts (dithiasuccinoyl), Rdtc (R=Alkyl or Aryl, dtc=dithiocarbamate), DBFmoc (2,7-di-t-butylFmoc or 1,7-di-t-butylfluoren-9-ylmethoxycarbonyl), Alloc (allyloxycarbonyl), pNZ (p-nitrobenzyloxycarbonyl), Nsc ([[2-[(4-nitrophenyl)sulfonyl]ethoxy]carbonyl]), Msc (2-methylsulfonylethoxycarbonyl), and MBz (4-methoxyCBz) are also useful N-terminus histidine protecting groups with respect to solid phase synthesis and solution or solid phase coupling of the peptide fragment. Bpoc [(1-[1,1′-biphenyl]-4-yl-1-methylethoxy)carbonyl], Bnpeoc [[2,2-bis(4-nitrophenyl)ethoxy]carbonyl], CBz [(phenylmethoxy)carbonyl], Aoc [(1,1-dimethylpropoxy)carbonyl], and Moz [[(4-methoxyphenyl)methoxy]carbonyl] are useful N-terminus histidine protecting groups with respect to solid phase synthesis and solution or solid phase coupling of the peptide fragment.

Fragment 2 includes the 10 amino acid residues corresponding to the amino acids in the 18 through 27 positions of the native GLP-1(7-36) peptide, and therefore may be represented by the notation GLP-1(18-27) and is a fragment according to SEQ ID NO. 12:

SYLEGQAAKE

Solid phase synthesis is generally carried out in a direction from the C-terminus to the N-terminus of the fragment 2. Thus, the E²⁷ amino acid, which is present on the C-terminal portion of the fragment, is the first amino acid residue that is coupled to the solid phase resin support. Solid phase synthesis then proceeds by consecutively adding amino acid residues in a manner corresponding to the desired sequence. The synthesis of the peptide intermediate fragment is complete after the N-terminal residue (for example, the N-terminal serine residue (S) has been added to the nascent peptide chain.

Fragment 3′ is a peptide fragment, or counterpart thereof, including amino acid residues according to SEQ ID NO. 14 wherein X³⁵ is as defined above, or is a

FIAWLVKX³⁵R

counterpart thereof including the X³⁵ residue. One or more of the amino acid residues may include side chain protecting groups in accordance with conventional practices. Fragment 3′ includes the amino acid residues corresponding to the amino acids in the 23 through 36 positions of the native GLP-1(7-36) peptide, except that X³⁵ is at the 35 position in place of the native amino acid at that position. Fragment 3′ may be represented by the notation (X³⁵)GLP-1(23-36). Fragment 3′ is conveniently prepared

FIAWLVKX³⁵

from fragment 3 (SEQ ID NO. 15). Fragment 3 is prepared by solid phase synthesis from Fmoc-Aib³⁵-O-2CT using standard coupling protocols. The lysine and tryptophan side chains were protected with Boc. The glutamic acid side chain was protected as a tert-Bu ester and the glutamine side chain was protected by a trityl group. Fragment 3 was cleaved from the resin and coupled with H-Arg (2HCl)—NH₂. Fragment 3 includes the amino acid residues corresponding to amino acids in positions 28 through 35 of native GLP-1(7-36) except that X³⁵ is Aib.

In some embodiments, the peptide fragment 3 may be resin bound via the C-terminus. This fragment optionally may bear side chain, N-terminus and/or C-terminus protection groups. Fmoc has been found to be a particularly useful N-terminus protecting group with respect to solid phase synthesis of the peptide fragment. In preferred embodiments, X³⁵ is Aib or a counterpart thereof including the Aib at the 35 position and may be represented by the notation (Aib³⁵)GLP-1(28-35) to note the substitution of Aib for the native amino acid at the 35 position of the native GLP-1(7-35).

Due to steric hindrance proximal to the X³⁵-loaded support resin, the coupling of lysine (34) and valine (33) onto the growing peptide chain can be problematic. Even with an excess of amino acid, it is difficult to force these coupling reactions to completion. Solvent choice and/or end-capping can help to alleviate this problem. It has been found that the nature of the coupling solvent can impact the degree to which the coupling goes to completion. In one set of experiments, for example, coupling reactions were carried out in a 3:1 NMP/DCM, 1:1 NMP/DCM, 1:1 DMF/DCM, and 3:1 DMF/DCM. The ratios in these solvent combinations are on a volume basis. NMP refers to N-methyl pyrrolidone, DCM refers to dichloromethane, and DMF refers to dimethylformamide. It was found that the coupling reactions proceeded farther to completion when using 1:1 DMF/DCM.

End-capping after each of the lysine and valine couplings can also be used to prevent unreacted resin-supported material from proceeding in further coupling reactions. The end-capped material is more easily removed during purification if desired. Conventional end-capping techniques may be used.

Continuing to refer to Scheme 2, fragments 1, 2, and 3′, are assembled to complete the desired peptide.

Fragments 2 and 3′ are first coupled in solution to form fragment 2+3′, and is according to SEQ. ID NO. 7:

SYLEGQAAKEFIAWLVKX³⁵R-NH₂

which may be designated by the notation (X³⁵)GLP-1(18-36). Fragment 2+3′ is then coupled to fragment 1 in the solution phase. To the extent that the other amino acids bear side chain protection, this protection desirably is maintained through this step. The desired peptide, incorporating fragments 1+2+3′, according to SEQ ID NO. 9:

HX⁸EGTFTSDVSSYLEGQAAKEFIAWLVKX³⁵R-NH₂

is then formed, wherein, in a preferred embodiment, X⁸ and X³⁵ are Aib as defined above.

In carrying out the reaction schemes of Schemes 1 and 2, solid phase and solution phase syntheses may be carried out by standard methods known in the industry. In representative modes of practice, peptides are synthesized in the solid phase using chemistry by which amino acids are added from the C-terminus to the N-terminus. Thus, the amino acid or peptide group proximal to the C-terminus of a particular fragment is the first to be added to the resin. This occurs by reacting the C-terminus functionality of the amino acid or peptide group with complementary functionality on the resin support. The N-terminus side of the amino acid or peptide group is masked to prevent undesired side reactions. The amino acid or peptide group desirably also includes side chain protection as well. Then successive amino acids or peptide groups are attached to the support-bound peptide material until the peptide of interest is formed. Most of these also include side chain protection in accordance with conventional practices. With each successive coupling, the masking group at the N-terminus end of the resin bound peptide material is removed. This is then reacted with the C-terminus of the next amino acid or peptide group whose N-terminus is masked. The product of solid phase synthesis is thus a peptide bound to a resin support.

Any type of support suitable in the practice of solid phase peptide synthesis can be used. In preferred embodiments, the support comprises a resin that can be made from one or more polymers, copolymers or combinations of polymers such as polyamide, polysulfamide, substituted polyethylenes, polyethyleneglycol, phenolic resins, polysaccharides, or polystyrene. The polymer support can also be any solid that is sufficiently insoluble and inert to solvents used in peptide synthesis. The solid support typically includes a linking moiety to which the growing peptide is coupled during synthesis and which can be cleaved under desired conditions to release the peptide from the support. Suitable solid supports can have linkers that are photo-cleavable, TFA-cleavable, HF-cleavable, fluoride ion-cleavable, reductively-cleavable; Pd(O)-cleavable; nucleophilically-cleavable; or radically-cleavable. Preferred linking moieties are cleavable under conditions such that the side-chain groups of the cleaved peptide are still substantially globally protected.

In one preferred method of synthesis, the peptide intermediate fragments synthesized on an acid sensitive solid support that includes trityl groups, and more preferably on a resin that includes trityl groups having pendent chlorine groups, for example a 2-chlorotrityl chloride (2-CTC) resin (Barlos et al. (1989) Tetrahedron Letters 30(30):3943-3946). Examples also include trityl chloride resin, 4-methyltrityl chloride resin, 4-methoxytrityl chloride resin. Some preferred solid supports include polystyrene, which can be copolymerized with divinylbenzene, to form support material to which the reactive groups are anchored.

Other resins that are used in solid phase synthesis include “Wang” resins, which comprise a copolymer of styrene and divinylbenzene with 4-hydroxymethylphenyloxymethyl anchoring groups (Wang, S. S. 1973, J. Am. Chem. Soc.), and 4-hydroxymethyl-3-methoxyphenoxybutyric acid resin (Richter et al. (1994), Tetrahedron Letters 35(27):4705-4706). The Wang, 2-chlorotrityl chloride, and 4-hydroxymethyl-3-methoxyphenoxy butyric acid resins can be purchased from, for example, Calbiochem-Novabiochem Corp., San Diego, Calif.

In order to prepare a resin for solid phase synthesis, the resin can be pre-washed in suitable solvent(s). For example, a solid phase resin such as a 2-CTC resin is added to a peptide chamber and pre-washed with a suitable solvent. The pre-wash solvent may be chosen based on the type of solvent (or mixture of solvents) that is used in the coupling reaction, or vice versa. Solvents that are suitable for washing, and also the subsequent coupling reaction include dichloromethane (DCM), dichloroethane (DCE), dimethylformamide (DMF), and the like, as well as mixtures of these reagents. Other useful solvents include DMSO, pyridine, chloroform, dioxane, tetrahydrofuran, ethyl acetate, N-methylpyrrolidone, and mixtures thereof. In some cases coupling can be performed in a binary solvent system, such as a mixture of DMF and DCM at a volume ratio in the range of 9:1 to 1:9, more commonly 4:1 to 1:4.

The syntheses of the present invention preferably are carried out in the presence of appropriate protecting groups unless otherwise noted. The nature and use of protecting groups is well known in the art. Generally, a suitable protecting group is any sort of group that that can help prevent the atom or moiety to which it is attached, e.g., oxygen or nitrogen, from participating in undesired reactions during processing and synthesis. Protecting groups include side chain protecting groups and amino- or N-terminal protecting groups. Protecting groups can also prevent reaction or bonding of carboxylic acids, thiols and the like.

A side chain protecting group refers to a chemical moiety coupled to the side chain (i.e., R group in the general amino acid formula H₂N—C(R)(H)—COOH) of an amino acid that helps to prevent a portion of the side chain from reacting with chemicals used in steps of peptide synthesis, processing, etc. The choice of a side chain-protecting group can depend on various factors, for example, type of synthesis performed, processing to which the peptide will be subjected, and the desired intermediate product or final product. The nature of the side chain protecting group also depends on the nature of the amino acid itself. Generally, a side chain protecting group is chosen that is not removed during deprotection of the α-amino groups during the solid phase synthesis. Therefore the α-amino protecting group and the side chain protecting group are typically not the same.

In some cases, and depending on the type of reagents used in solid phase synthesis and other peptide processing, an amino acid may not require the presence of a side-chain protecting group. Such amino acids typically do not include a reactive oxygen, nitrogen, or other reactive moiety in the side chain.

Examples of side chain protecting groups include acetyl (Ac), benzoyl (Bz), tert-butyl, triphenylmethyl (trityl), tetrahydropyranyl, benzyl ether (Bzl) and 2,6-dichlorobenzyl (DCB), t-butoxycarbonyl (Boc), nitro, p-toluenesulfonyl (Tos), adamantyloxycarbonyl, xanthyl (Xan), benzyl, 2,6-dichlorobenzyl, methyl, ethyl and t-butyl ester, benzyloxycarbonyl (cBz or Z), 2-chlorobenzyloxycarbonyl (2-Cl-Z), t-amyloxycarbonyl (Aoc), and aromatic or aliphatic urethan-type protecting groups. photolabile groups such as nitro-veratryloxycarbonyl (NVOC); and fluoride labile groups such as 2-trimethylsilylethoxycarbonyl (TEOC).

Preferred side chain protecting groups for amino acids commonly used to synthesize GLP-1 peptides in the practice of the present invention are shown in the following Table A:

TABLE A

| Amino Acid | Side Chain Protecting group(s) |
|---|---|
| Aib | None |
| Ala | None |
| Arg | None |
| Asp | t-butyl ester (OtBu) |
| Gln | trityl (trt) |
| Glu | OtBu |
| Gly | None |
| His | trityl (trt) |
| Ile | None |
| Leu | None |
| Lys | t-butyloxycarbonyl (Boc) |
| Phe | None |
| Ser | t-butyl (tBu) |
| Thr | tBu |
| Trp | Boc |
| Tyr | tBu |
| Val | None |

An amino-terminal protecting group includes a chemical moiety coupled to the alpha amino group of an amino acid. Typically, the amino-terminal protecting group is removed in a deprotection reaction prior to the addition of the next amino acid to be added to the growing peptide chain, but can be maintained when the peptide is cleaved from the support. The choice of an amino terminal protecting group can depend on various factors, for example, type of synthesis performed and the desired intermediate product or final product.

Examples of amino-terminal protecting groups include (1) acyl-type protecting groups, such as formyl, acrylyl (Acr), benzoyl (Bz) and acetyl (Ac); (2) aromatic urethane-type protecting groups, such as benzyloxycarbonyl (Z) and substituted Z, such as p-chlorobenzyloxycarbonyl, p-nitrobenzyloxycarbonyl, p-bromobenzyloxycarbonyl, p-methoxybenzyloxycarbonyl; (3) aliphatic urethan protecting groups, such as t-butyloxycarbonyl (Boc), diisopropylmethoxycarbonyl, isopropyloxycarbonyl, ethoxycarbonyl, allyloxycarbonyl; (4) cycloalkyl urethan-type protecting groups, such as 9-fluorenyl-methyloxycarbonyl (Fmoc), cyclopentyloxycarbonyl, adamantyloxycarbonyl, and cyclohexyloxycarbonyl; and (5) thiourethan-type protecting groups, such as phenylthiocarbonyl. Preferred protecting groups include 9-fluorenyl-methyloxycarbonyl (Fmoc), 2-(4-biphenylyl)-propyl(2)oxycarbonyl (Bpoc), 2-phenylpropyl(2)-oxycarbonyl (Poc) and t-butyloxycarbonyl (Boc).

Fmoc or Fmoc-like chemistry is highly preferred for solid phase peptide synthesis, inasmuch as cleaving the resultant peptide in a protected state is relatively straightforward to carry out using mildly acidic cleaving agents. This kind of cleaving reaction is relatively clean in terms of resultant by-products, impurities, etc., making it technically and economically feasible to recover peptide on a large scale basis from both the swelling and shrinking washes, enhancing yield. As used herein, “large scale” with respect to peptide synthesis generally includes the synthesis of peptides in the range of at least 500 g, more preferably at least 2 kg per batch. Large-scale synthesis is typically performed in large reaction vessels, such as steel reaction vessels, that can accommodate quantities of reagents such as resins, solvents, amino acids, chemicals for coupling, and deprotection reactions, that are sized to allow for production of peptides in the kilogram to metric ton range.

Additionally, the Fmoc protecting group can be selectively cleaved from a peptide relative to the side chain protecting groups so that the side chain protection are left in place when the Fmoc is cleaved. This kind of selectivity is important during amino acid coupling to minimize side chain reactions. Additionally, the side chain protecting groups can be selectively cleaved to remove them relative to the Fmoc, leaving the Fmoc in place. This latter selectivity is very advantageously relied upon during purification schemes described further below.

The solid phase coupling reaction can be performed in the presence of one or more compounds that enhance or improve the coupling reaction. Compounds that can increase the rate of reaction and reduce the rate of side reactions include phosphonium and uronium salts that can, in the presence of a tertiary base, for example, diisopropylethylamine (DIEA) and triethylamine (TEA), convert protected amino acids into activated species (for example, BOP, PyBOP, HBTU, and TBTU, which generate HOBt esters, and DEPBT which generates an HOOBt ester). Other reagents help prevent racemization by providing a protecting reagent. These reagents include carbodiimides (for example, DCC or WSCDI) with an added auxiliary nucleophile (for example, 1-hydroxy-benzotriazole (HOBt), 1-hydroxy-azabenzotriazole (HOAt), or HOSu). The mixed anhydride method, using isobutyl chloroformate, with or without an added auxiliary nucleophile, may also be utilized, as can the azide method, due to the low racemization associated with it. These types of compounds can also increase the rate of carbodiimide-mediated couplings, as well as prevent dehydration of Asn and Gln residues.

After the coupling is determined to be complete, the coupling reaction mixture is washed with a solvent, and the coupling cycle is repeated for each of the subsequent amino acid residues of the peptide material. In order to couple the next amino acid, removal of the N-terminal protecting group (for example, an Fmoc group) from the resin-bound material is typically accomplished by treatment with a reagent that includes 10-50% (on a weight basis) piperidine in a solvent, such as N-methylpyrrolidone (NMP) or dimethylformamide (DMF). After removal of the Fmoc protecting group, several washes are typically performed to remove residual piperidine and Fmoc by-products (such as dibenzofulvene and its piperidine adduct).

The subsequent amino acids can be utilized at a stoichiometric excess of amino acids in relation to the loading factor of peptide material on the resin support. Generally, the amount of amino acids used in the coupling step is at least equivalent to the loading factor of the first amino acid on the resin (1 equivalent or more). Preferably the amount of amino acids used in the coupling step is 1.7 to 2.0 equivalents.

Following the final coupling cycle, the resin is washed with a solvent such as NMP, and then washed with an inert second solvent such as DCM. In order to remove the synthesized peptide material from the resin, a cleaving treatment is carried out in a manner such that the cleaved peptide material still bears sufficient side chain and terminus protecting groups. Leaving the protective groups in place helps to prevent undesirable coupling or other undesirable reactions of peptide fragments during or after resin cleavage. In the case when Fmoc or similar chemistry is used to synthesize the peptide, protected cleavage may be accomplished in any desired fashion such as by using a relatively weak acid reagent such as acetic acid or dilute TFA in a solvent such as DCM. The use of 0.5 to 10 weight percent, preferably 1 to 3 weight percent TFA in DCM is typical. See, e.g., U.S. Pat. No. 6,281,335.

Steps of cleaving the peptide intermediate fragment from the solid phase resin can proceed along the lines of an exemplary process as follows. However, any suitable process that effectively cleaves the peptide intermediate fragment from the resin can be used. For example, approximately 5 to 20, preferably about 10 volumes of a solvent containing an acidic cleaving reagent is added to the vessel containing the resin-bound peptide material. The resin, typically in the form of beads, is immersed in the reagent as a consequence. The cleaving reaction occurs as the liquid contents are agitated at a suitable temperature for a suitable time period. Agitation helps prevent the beads from clumping. Suitable time and temperature conditions will depend upon factors such as the acid reagent being used, the nature of the peptide, the nature of the resin, and the like. As general guidelines, stirring at from about −15° C. to about 5° C., preferably from about −10° C. to about 0° C. for about 5 minutes to two hours, preferably about 25 minutes to about 45 minutes would be suitable. Cleaving time may be in the range of from about 10 minutes to about 2 hours or even as much as a day. Cleaving is desirably carried out in such a chilled temperature range to accommodate a reaction exotherm that might typically occur during the reaction. In addition, the lower temperature of the cleavage reaction prevents acid sensitive side chain protecting groups, such as trt groups, from being removed at this stage.

At the end of the cleaving treatment, the reaction is quenched. This may be achieved, for example, by combining the cleaving reagent with a suitable base, such as pyridine or the like, and continuing to agitate and stir for an additional period such as for an additional 5 minutes to 2 hours, preferably about 20 minutes to about 40 minutes. Adding the base and continued agitation causes the temperature of the vessel contents to increase. At the end of agitation, the vessel contents may be at a temperature in the range of from about 0° C. to about 15° C., preferably about 5° C. to about 10° C.

Factors such as swelling and shrinking the resin in order to improve aspects of the peptide recovery can optionally be incorporated into the overall synthesis process. These techniques are described, for example, in U.S. Pat. Pub. No. 2005/0164912 A1.

In some aspects, the cleaved peptide fragments can be prepared for solution phase coupling to other peptide fragments and/or amino acids. Peptide coupling reactions in the solution phase are reviewed in, for example, New Trends in Peptide Coupling Reagents; Albericio, Fernando; Chinchilla, Rafeal; Dodsworth, David J.; and Najera, Armen; Organic Preparations and Procedures International (2003), 33(3), 203-303.

Coupling of peptide intermediate fragments to other fragments or amino acid(s) in the solution phase can be carried out using in situ coupling reagents, for example benzotriazol-1-yl-oxy-tris-(dimethylamino)phosphoniumhexafluorophosphate (BOP), benzotriazol-1-yl-oxy-tripyrrolidinophosphonium hexafluorophosphate (PyBOP), o-(benzotriazol-1-yl)-N,N,N′,N′-tetramethyluronium hexafluorophosphate (HBTU), o-(7-azabenzotriazol-1-yl)-1,1,3,3-tetramethyluronium hexafluoroborate (HATU), o-(7-azabenzotriazol-1-yl)-1,1,3,3-tetramethyluronium tetrafluorophosphate (TATU), o-(1H-6-chloro-benzotriazol-1-yl)-1,1,3,3-tetramethyluronium hexafluorophosphate (HCTU), o-(1H-6-chloro-benzotriazol-1-yl)-1,1,3,3-tetramethyluronium tetrafluoroborate (TCTU), o-(benzotriazol-1-yl)oxybios-(pyrrolidino)-uronium hexafluorophosphate (HAPyU), dicyclohexylcarbodiimide (DCC), diisopropylcarbodiimide, 3-(diethoxyphosphoryloxy)-1,2,3-benzotriazine-4(3H)-one (DEPBT), water-soluble carbodiimide (WSCDI), o-(cyano-ethoxycarbonyl-methyleneamino)-N,N,N′,N″-tetramethyluronium tetrafluoroborate (TOTU) or o-(benzotriazol-1-yl)-N,N,N′,N′-tetramethyluronium tetrafluoroborate (TBTU). Other coupling techniques use preformed active esters such as hydroxysuccinimide (HOSu) and p-nitrophenol (HONp) esters; preformed symmetrical anhydrides; non-symmetrical anhydrides such as N-carboxyanhydrides (NCAs); or acid halides such as acyl fluoride as well as acyl chloride.

A suitable coupling solvent can be used in the solution phase coupling reaction. It is understood that the coupling solvent(s) used can affect the degree of racemization of the peptide bond formed; the solubility of the peptide and/or peptide fragments; and the coupling reaction rate. In some embodiments, the coupling solvent includes one or more water-miscible reagents. Examples of water-miscible solvents include, for example, DMSO, pyridine, chloroform, dioxane, tetrahydrofuran, ethyl acetate, N-methylpyrrolidone, dimethylformamide, dioxane, or mixtures thereof.

In other embodiments, the coupling reaction may include one or more non water-miscible reagents. An exemplary non water-miscible solvent is methylene chloride. In these embodiments, the non water-miscible solvent is preferably compatible with the deprotection reaction; for example, if a non water-miscible solvent is used preferably it does not adversely affect the deprotection reaction.

After the peptide of SEQ ID No. 9 is formed, the product can be subject to deprotection, purification, lyophilization, further processing (e.g., reaction with another peptide to form a fusion protein); combinations of these, and/or the like, as desired.

For example, according to the invention, the side-chain protecting groups are typically retained on the peptide intermediate fragments throughout solid phase synthesis and also into and throughout the solution phase coupling reactions. Generally, after solution phase step is completed, one or more deprotection steps may be performed to remove one or more protecting groups from the peptide. The removal of side chain protecting groups by global deprotection typically utilizes a deprotection solution that includes an acidolytic agent to cleave the side chain protecting groups. Commonly used acidolytic reagents for global deprotection include neat trifluoroacetic acid (TFA), HCl, Lewis acids such as BF₃Et₂O or Me₃SiBr, liquid hydrofluoric acid (HF), hydrogen bromide (HBr), trifluoromethanesulfonic acid, and combinations thereof. The deprotection solution also includes one or more suitable cation scavengers, for example, dithiothreitol (DTT), anisole, p-cresol, ethanedithiol, or dimethyl sulfide. The deprotection solution can also include water. As used herein, amounts of reagents present in the deprotection composition are typically expressed in a ratio, wherein the amount of an individual component is expressed as a numerator in “parts”, such as “parts weight” or “parts volume” and the denominator is the total parts in the composition. For example, a deprotection solution containing TFA:H₂O:DTT in a ratio of 90:5:5 (weight/weight/weight) has TFA at 90/100 parts by weight, H₂O at 5/100 parts by weight, and DTT at 5/100 parts by weight.

The precipitation is typically done using an ether, e.g., diethyl ether or MTBE (methyl tert-Bu ether). After precipitation, the peptide is desirably isolated and dried before being combined with other ingredients, lyophilized, packaged, stored, further processed, and/or otherwise handled. This may be accomplished in any suitable fashion. According to one suitable approach, the peptide is collected via filtering, washed with ample MTBE washes to reduce final salt content to a suitable level, and then dried.

The present invention also provides useful techniques for purifying a wide range of peptides, including GLP-1 peptides and their counterparts.

A particularly preferred purification process involves at least two purification passes through chromatographic media, wherein at least a first pass occurs at a first pH and at least a second pass occurs at a second pH. More preferably, the first pass occurs at an acidic pH, while the second pass occurs at a basic pH. In preferred embodiments, at least one pass under acidic conditions occurs prior to a pass occurring under basic conditions. An illustrative mode of practicing this purification approach can be described in the illustrative context of purifying fully protected peptide 11. Initially, the peptide is globally de-protected. Both N-terminus and side chain protecting groups are cleaved. A first chromatographic pass is carried out in a water/ACN gradient, using enough TFA to provide a pH of about 1 to 5, preferably about 2. A second pass is then carried out in a water/ACN gradient using a little ammonia and/or ammonium acetate, or the like, to provide a pH of around 8 to 9, preferably 8.5 to 8.9.

The pH values, whether acid or base, promote uniformity in that a uniform ionic species is present in each instance. Thus, the acidic pH desirably is sufficiently low so that substantially all of the amino acid residues in the peptide material are protonated. The basic pH is desirably high enough so that substantially all of the amino acid residues in the peptide material are deprotonated. The acid and base chromatography can be carried out in any order. It is convenient to do the basic chromatography last when the peptide acetate is a desired product inasmuch as the acetate may be the product of chromatography.

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc), di-tent-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), (3-(diethoxyphosphoryloxy)-1,2,3-benzotriazin-4(3H)-one) (DEPBT), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), ethylene glycol dimethyl ether (DME), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N, N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyl disilazane (LiHMDS), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), (benzotriazol-1-yloxy)tripyrrolidinophosphonium hexafluorophosphate (PyBOP), room temperature (rt or RT), tert-butyldimethylsilyl or t-BuMe₂Si (TBDMS), triethylamine (TEA or Et₃N), 2,2,6,6-tetramethylpiperidine 1-oxyl (TEMPO), triflate or CF₃SO₂— (Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me—C₆H₄SO₂— or tosyl (Ts), N-urethane-N-carboxyanhydride (UNCA). Conventional nomenclature including the prefixes normal (n), iso (i-), secondary (sec-), tertiary (tent-) and neo have their customary meaning when used with an alkyl moiety. (J. Rigaudy and D. P. Klesney, Nomenclature in Organic Chemistry, IUPAC 1979 Pergamon Press, Oxford.).

The principles of the present invention will now be further illustrated with respect to the following illustrative examples. In the following all percentages and ratios are by volume unless otherwise expressly stated.

### examples

### GLP-1 Solid Phase Synthesis of GLP-1 Fragment Fmoc-AA(7-17)-OH

Fmoc-His(trt)-Aib-Glu(OtBu)-Gly-Thr(tBu)-Phe-
Thr(tBu)-Ser(OtBu)-Asp(OtBu)-Val-Ser(OtBu)-OH

### Example 1

### Solid Phase Synthesis of the Fmoc-AA(7-17)-O-2CT2CT

Solid phase synthesis of Fmoc-AA(7-17)-OH was carried out starting with 15.0 g of H-Ser(OtBu)-2-CT resin (Peptides International; Lot#601511) loaded at 0.55 mmole/g. The resin was swelled in DCM (150 mL) for 30 min at 25° C. The DCM solvent was drained and the resin was washed three times with NMP (90 mL for each wash).

To prepare the coupling solution, the amino acid (2.0 equiv.) and 1-hydroxybenzotriazole hydrate (HOBT, 2.0 equiv.) were weighed, dissolved in 43.3 mL DMF then activated by combining with an HBTU (2.0 equiv.) solution in DMF (concentration: 205.76 g/L; 31.4 mL) and then adding DIEA (3.5 equiv.) at 0°-5° C. The resulting solution was added to reaction vessel containing resin, the activation flask was rinsed with 24.5 mL DCM into reactor, which was then stirred for 4-6 hours at 25° C. After 4 hours stirring coupling reaction mixture, the coupling solution was drained and the resin was washed with DMF 4 times (90 mL each wash). The resin was then treated twice with 20% Piperidine in DMF (90 mL each treatment) to remove Fmoc protecting groups. After the second 20% Piperidine/DMF treatment, the resin was washed nine times with DMF (90 mL each wash).The removal of the Fmoc protecting group and coupling reaction cycles were repeated for the remaining amino acids in the fragment (i.e., in the order of Val→Asp(OtBu)→Ser(tBu)→Thr(tBu)→Phe→Thr(tBu)→Gly→Glu(OtBu)→Aib→His(trt). The solvent for the final His(trt) coupling reaction was replaced DMF with 0.1 M LiBr in THF/NMP (3:1). And coupling reagent for the final His coupling reaction was used DEPBT as solution in DMF (concentration: 162.33 g/L; 31.4 mL). And His was recoupled one more time to ensure the coupling completion.

All reagents used in this example are listed in following table:

Coupling Reaction of the GPA Fmoc-AA(7-17)-OH Example 1

| Amino | | HOBT | DIEA | DMF | HBTU | DMF | DCM | Coupling |
|---|---|---|---|---|---|---|---|---|
| Acid | g | H₂O (g) | (mL) | (mL) | (g) | (mL) | (mL) | time (min) |
| Val | 5.62 | 2.53 | 5.0 | 43.3 | 5.10 | 25.2 | 24.5 | 300 |
| Asp(OtBu) | 6.81 | 2.51 | 5.0 | 43.3 | 5.10 | 25.2 | 24.5 | 300 |
| Ser(OtBu) | 6.35 | 2.52 | 5.0 | 43.3 | 5.10 | 25.2 | 24.5 | 300 |
| Thr(tBu) | 6.56 | 2.53 | 5.0 | 43.3 | 5.10 | 25.2 | 24.5 | 300 |
| Phe | 6.41 | 2.52 | 5.0 | 43.3 | 5.10 | 25.2 | 24.5 | 300 |
| Thr(tBu) | 6.59 | 2.53 | 5.0 | 43.3 | 5.10 | 25.2 | 24.5 | 300 |
| Gly | 4.95 | 2.52 | 5.0 | 43.3 | 5.10 | 25.2 | 24.5 | 300 |
| Glu(OtBu) | 7.36 | 2.52 | 5.0 | 43.3 | 5.10 | 25.2 | 24.5 | 300 |
| Aib | 5.40 | 2.53 | 5.0 | 43.3 | 5.10 | 25.2 | 24.5 | 300 |
| | | | | 0.1 M LiBr | DEPBT | 0.1 M LiBr | | |
| | | | | in THF/ | | in THF/ | | |
| | | | | NMP (3:1) | | NMP (3:1) | | |
| His(trt) | 10.26 | 2.52 | 5.0 | 42.0 | 6.46 | 26.5 | 24.5 | 240 |
| His(trt) | 10.22 | 2.52 | 5.0 | 42.0 | 6.46 | 26.5 | 24.5 | 300 |

The built resin was washed with NMP (90 mL.) 4 times and DCM (90 mL.) 7 times.

Cleavage of the GLP-1 Fragment Fmoc-AA(7-17)-OH from Built Resin

The built resin from above was cooled with the last DCM wash to −5° C. The DCM was drained and the cold solution of 1% v/v TFA/DCM (150 mL at −5° to −10° C.) was added and stirred at 0° C. Pyridine (4.0 mL) was added to the cleavage receiver for the neutralization of the TFA. After 15 min treating the built resin at 0° C., the cleavage solution was collected in the cleavage receiver. Another cold solution of 1% TFA/DCM (150 mL at −5° to −10° C.) was added and stirred for 15 min at 0° C. then drain to the cleavage receiver. The third cold solution of 1% TFA/DCM (150 mL at −5° to −10° C.) was added and stirred for 30 min at 0° C. then Pyridine (2.0 mL) was added to cleavage vessel to neutralize TFA and also drain the final cleavage solution to the cleavage receiver. While vessel warming up to 25° C., the resin was washed with DCM 5 times (90 mL) and drained into the cleavage solution receiver. The combining DCM cleavage and wash solution were concentrated to 90 mL and then combined with water (90 mL.). The DCM was distilled under reduced pressure with vigorously agitation (350-50 torr at 25° C.). The fragment precipitated out from the water mixture when the DCM was removed. The product then was filtered, washed with water, and vacuum dried at 35° C.A total of 14.371 g of Fmoc-AA(7-17)-OH with a purity of 95.7% AN was obtained, yield of 90.7%.

### GLP-1 Solid Phase Synthesis of GLP-1 Fragment Fmoc-AA(18-22)-OH

Fmoc-Ser(OtBu)-Tyr(tBu)-Leu-Glu(OtBu)-Gly-OH

### Example 2

### Solid Phase Synthesis of the Fmoc-AA(18-22)-O-2CT2CT

Solid phase synthesis of Fmoc-AA(11-22)-OH was carried out starting with 20.0 g of H-Gly-2-CT resin (Patras; Lot#2592) loaded at 0.51 mmole/g. The resin was swelled in DCM (200 mL) for 30 min at 25° C. The DCM solvent was drained and the resin was washed three times with NMP (120 mL) three times.

To prepare the coupling solution, the amino acid (2.0 equiv.) and 1-hydroxybenzotriazole hydrate (HOBT.H2O; 0.16 g; 0.1 equiv.) were weighed, dissolved in 29.8 mL of NMP then activated by combining with an HBTU solution in NMP (concentration: 172.4 g/L; 43.8 mL; 1.95 equiv.) and then adding DIEA (3.9 mL; 2.2 equiv.) at 0°-5° C. The resulting solution was added to reaction vessel containing resin, the activation flask was rinsed with 23.7 mL DCM into reactor, which was then stirred at 22° C. After 5.0-6.5 hours stirring coupling reaction mixture, the coupling solution was drained and the resin was washed with NMP 4 times (120 mL). The resin was then treated twice with 20% Piperidine in NMP (120 mL) to remove Fmoc protecting groups. After the second 20% Piperidine/NMP treatment, the resin was washed nine times with NMP (120 mL). The removal of the Fmoc protecting group and coupling reaction cycles were repeated for the remaining amino acids in the fragment (i.e., in the order of Glu(OtBu)→Leu→Tyr(tBu)→Ser(tBu).

All reagents used in this example are listed in following table:

Coupling Reaction of the GPA Fmoc-AA(18-22)-OH Example 2

| | | HOBT | | | | | | Coupling |
|---|---|---|---|---|---|---|---|---|
| | | H₂O | DIEA | NMP | HBTU | NMP | | time |
| Amino Acid | g | (g) | (mL) | (mL) | (g) | (mL) | DCM (mL) | (min) |
| Glu(OtBu) | 9.06 | 0.18 | 3.9 | 29.8 | 7.55 | 36.2 | 23.7 | 390 |
| Leu | 7.24 | 0.17 | 3.9 | 29.8 | 7.55 | 36.2 | 23.7 | 300 |
| Tyr(tBu) | 9.38 | 0.17 | 3.9 | 29.8 | 7.55 | 36.2 | 23.7 | 300 |
| Ser(tBu) | 7.84 | 0.16 | 3.9 | 29.8 | 7.55 | 36.2 | 23.7 | 360 |

The built resin was washed with NMP (120 mL.) 4 times and DCM (120 mL.) 7 times.

Cleavage of the GLP-1 Fragment Fmoc-AA(18-22)-OH from Built Resin

The built resin from above was cooled with the last DCM wash to −5° C. The DCM was drained and the cold solution of 1% v/v TFA/DCM (160 mL at −5° to −10° C.) was added and stirred at 0° C. Pyridine (2.0 mL) was added to the cleavage receiver for the neutralization of the TFA from the first cleavage solution. After 30 min stirring, the cleavage solution was collected in the cleavage receiver. Then another cold solution of 1% TFA/DCM (160 mL at −5° to −10° C.) was added and stirred for 30 min at 0° C. Pyridine (2.1 mL) was added to cleavage vessel to neutralize TFA. After drain the second cleavage solution into cleavage receiver, vessel warming up to 25° C., the resin was washed with DCM 6 times (120 mL.) and drained into the cleavage solution receiver. The combining DCM cleavage and wash solution were concentrated to a volume of 150 mL and then combined with water (150 mL). The DCM was distilled under reduced pressure with vigorously agitation (350-50 torr at 25° C.). The fragment precipitated out from the water mixture when the DCM was removed. The fragment was washed with water and dried at 30°-35° C. under vacuum. A total of 8.76 g of GL-1 Fmoc-AA(18-22)-OH with a purity of 98.6% AN was obtained, yield of 89.6%.

### The Solution Phase Synthesis of the GLP-1 Fragment H-AA(18-36)-NH₂

H-Ser(OtBu)-Tyr(tBu)-Leu-Glu(OtBu)-Gly-Gln(trt)-
Ala-Ala-Lys(Boc)-Glu(OtBu)-Phe-Ile-Ala-Trp(Boc)-
Leu-Val-Lys(Boc)-Aib-Arg-NH2

### Example 3

The GPA Fragment 3′ H-AA(23-36)-NH2 (2.76 g), Fragment Fmoc-AA(18-22)-OH (0.99 g), and HOBT hydrate (0.16 g) were dissolved in DMF (14 mL). To this solution, a solution of BOP (0.56 g) in DMF (15 mL) and DIEA (0.29) were charged along with a DMF rinse (10 mL). The reaction was stirred at 20° C. and monitored by HPLC. After 4 hours, the coupling reaction was uncompleted. The kickers of fragment additional Fragment Fmoc-AA(18-22)-OH (0.02 g) , the solution BOP (0.07 equiv.) in 1 mL DMF, and DIEA (0.07.) were added along with a DMF rinse (1 mL). The reaction was complete after overnight agitation. Piperidine (0.38 g) was charged to the reaction mixture. The Fmoc removal was done after 1 hours at 38° C. After cooling to 25° C., the reaction mixture was quenched with water (100 mL) at ambient temperature. The quenched mixture was extracted with DCM (100 mL). The DCM layer was washed with water (2×100 mL), and concentrated to ˜20 g. The concentrated DCM solution was feed into a stirring Heptane to precipitate the product. Then the DCM in the precipitation mixture was distilled out under vacuum (350-50 mm Hg) at 20° to 25° C. then MTBE (100 mL) were charged and stirred overnight at 25° C. The solid was filtered and washed with MTBE/Heptane (1:1, 50 mL each) twice. The filter cake was air dried for 0.5 hours and then vacuum dried at 35°-40° C. A total of 3.37 g, 96.6% actual yield, was obtained with a purity of 81.9% AN.

### Example 4

The GPA Fragment 3′ H-AA(23-36)-NH₂ (synthesiszed generally according to procedures in U.S. Ser. No. 12/316,309) (total 2.83 g) was dissolved in DMF (24 mL) & Methyl-THF (5 mL) at 35°-40° C. for 3 hours, then cool to 0°-5° C. Then the cold (0°-5° C.) solution of the Fragment Fmoc-AA(18-22)-OH (1.154 g) and HOBT.hydrate (0.018 g) in the mixed solvents of DMF (2 mL) & Me-THF (24 mL) was charged along with a DMF rinse (5 mL). To this resulting solution, a solution of BOP (0.69 g) in DMF (2 mL) and DIEA (0.21 g) were charged along with a DMF rinse (10 mL). The reaction was stirred at 0° C. and monitored by HPLC. After 15.5 hours, the coupling reaction was uncompleted. The kickers of fragment additional Fragment 3′ (0.172 g), the solution BOP (0.16 g) in 2 mL DMF, and DIEA (0.15.) were added along with a DMF rinse (1 mL). The reaction was complete after overnight agitation at 20° C. Piperidine (0.44 g) was charged to the reaction mixture. The Fmoc removal was done after 1 hour at 38° C. After cooling to 25° C., the reaction mixture was quenched with water (75 mL) and Me-THF (30 mL) at ambient temperature. After phase separation, Me-THF (15 mL) was used to back extraction of the low aqueous layer. The combined Me-THF layers were concentrated on rotary evaporator then fresh Me-THF (30 mL) was charged to dissolve the residue. The concentration, redissolving in Me-THF (30 mL), and concentration operations were repeated one more time. The residue finally was dissolved in Me-THF (15 mL) and fed into the stirring Heptane (120 mL) along with a Me-THF (3 mL) rinse. The precipitated solid were filtered and washed with Heptane (25 mL each). The filter cake was air dried for 0.5 hours and then vacuum dried at 35°-40° C. A total of 3.66 g, 95.5% actual yield, was obtained with a purity of 85.1% AN.

### The Solution Phase Synthesis of the Crude GLP-1 Following Scheme 1

### Example 5

The GLP-1 Fragment Fmoc-AA(7-17)-OH (0.843 g) was dissolved in the THF (20 mL). Then combined this solution with Fragment H-AA(18-36)-NH₂) (1.433 g) and stirred to dissolve all solid. To this solution, 6-Cl-HOBt (0.101 g), DEPBT (0.199 g), and then DIEA (0.132 mL) was charged along with a THF rinse (3 mL). The reaction was agitated at room temperature (18°-22° C.) and monitored by HPLC. After 2 days, reaction completion check indicated that the reaction was not completed (11% excess of Fragment Fmoc-AA(7-17)-OH). The kickers of Fragment H-AA(18-36)-NHS (0.123 g), DEPBT (0.039 g), and DIEA (0.043 mL) were added and continue stirring at room temperature. After 19.5 hrs, HPLC analysis indicated that the reaction was completed. Piperidine (0.267 g) was charged and stirred the resulting reaction mixture at room temperature. After stirring for 7.5 hrs, the deproection reaction was done. The THF in the reaction mixture then was displaced with DCM (2×15 mL) under vacuum (35° C. under 130 mm Hg vacuum). The residue was dissolved in DCM (5.6 mL) and combined with a solution of the DTT (1.11 g), water (1.09 g), and TFA (19 mL) at 14° C. After stirring 6 hours at 15° C., the reaction mixture was quenched by charging cold (−5° C.) MTBE (89 mL). The quenched reaction mixture was aged at 15° for 30 min. The solid product was filtered, washed with MTBE (3×19 mL), and dried overnight under vacuum at 35° C. A 1.91 g of GPA crude (34.3% wt/wt) was obtained with a purity of 63.4% AN; a 45.6% yield.

### GLP-1 Solid Phase Synthesis of GLP-1 Fragment Fmoc-AA(19-27)-OH

Fmoc-Ser(OtBu)-Tyr(tBu)-Leu-Glu(OtBu)-Gly-
Gln(trt)-Ala-Ala-Lys(Boc)-Glu(OtBu)-OH

### Example 6

### Solid Phase Synthesis of the Fmoc-AA(19-27)-O-2CT2CT

Solid phase synthesis of Fmoc-AA(19-27)-OH was carried out starting with 20.0 g of Fmoc-Glu(OtBu)-2-CTC resin loaded at 0.58 mmole/g. The resin was swelled in DCM (200 mL) for 30 min at 25° C. The DCM solvent was drained and the resin was washed with DMF (120 mL) three times. The removal of Fmoc protection group was achieved by treating the swelled resin twice with 20% Piperidine in DMF (120 mL). After the second 20% Piperidine/DMF treatment, the resin was washed nine times with DMF (120 mL).

To prepare the coupling solution, the amino acid (2.0 equiv.) and 1-hydroxybenzotriazole hydrate (HOBT.H2O; 3.55 g; 2.0 equiv.) were weighed, dissolved in 60.0 mL of DMF then activated by combining with an HBTU solution in DMF (concentration: 205.76 g/L; 42.8 mL; 2.0 equiv.) and then adding DIEA (9.1 mL; 4.5 equiv.) at 0°-5° C. The resulting solution was added to reaction vessel containing resin, the activation flask was rinsed with 34.3 mL DCM into reactor, which was then stirred at 25° C. After 5.0 hours stirring coupling reaction mixture, the coupling solution was drained and the resin was washed with DMF 4 times (120 mL). The resin was then treated twice with 20% Piperidine in DMF (120 mL) to remove Fmoc protecting groups. After the second 20% Piperidine/DMF treatment, the resin was washed nine times with DMF (120 mL).The removal of the Fmoc protecting group and coupling reaction cycles were repeated for the remaining amino acids in the fragment (i.e., in the order of Lys(Boc)→Ala→Ala→Gln(trt)→Gly→Glu(OtBu)→Leu→Tyr(tBu)).

All reagents used in this example are listed in following table:

Coupling Reaction of the GPA Fmoc-AA(19-27)-O-2CT Example 1

| Amino | | HOBT | DIEA | DMF | HBTU | DMF | DCM | Coupling |
|---|---|---|---|---|---|---|---|---|
| Acid | g | H₂O (g) | (mL) | (mL) | (g) | (mL) | (mL) | time (min) |
| Lys(Boc) | 10.89 | 3.55 | 9.1 | 60.0 | 8.8 | 34.0 | 34.3 | 300 |
| Ala | 7.65 | 3.53 | 9.1 | 60.0 | 8.8 | 34.0 | 34.3 | 300 |
| Ala | 7.65 | 3.55 | 9.1 | 60.0 | 8.8 | 34.0 | 34.3 | 300 |
| Gln(trt) | 14.20 | 3.54 | 9.1 | 60.0 | | 34.0 | 34.3 | 300 |
| Gly | 6.93 | 3.54 | 9.1 | 60.0 | 8.8 | 34.0 | 34.3 | 300 |
| Glu(OtBu) | 9.90 | 3.53 | 9.1 | 60.0 | 8.8 | 34.0 | 34.3 | 300 |
| Leu | 8.19 | 3.56 | 9.1 | 60.0 | 8.8 | 34.0 | 34.3 | 300 |
| Tyr(tBu) | 10.68 | 3.56 | 9.1 | 60.0 | 8.8 | 34.0 | 34.3 | 300 |

The built resin was sequentially washed with DMF (120 mL.) 4 times, DCM (120 mL.) 8 times and IPA (120 mL) 4 times. Then the built resin was vacuum dried at 35° C. and led 32.75 g Fmoc-(19-27)-O-2CT Resin. An 83.6% yield is based on the weight gain of resin.

### Example 7

### Solid Phase Synthesis of the Fmoc-AA(18-27)-O-2CT2CT

Solid phase synthesis of Fmoc-AA(18-27)-OH was carried out with 16.38 g of Fmoc-AA(19-27)-O-2-CTC resin from above. The resin was swelled in DCM (100 mL) for 30 min at 25° C. The DCM solvent was drained and the resin was washed with DMF (60 mL) three times. The removal of Fmoc protection group was achieved by treating the swelled resin twice with 20% Piperidine in DMF (60 mL). After the second 20% Piperidine/DMF treatment, the resin was washed nine times with DMF (60 mL).

To prepare the coupling solution, the Fmoc-Ser(OtBu) (4.48 g, 2.0 equiv. based on Fmoc-Glu(OtBu)-O-2CT Resin) and 1-hydroxybenzotriazole hydrate (HOBT.H2O; 1.78 g; 2.0 equiv. based on Fmoc-Glu(OtBu)-O-2CT Resin) were weighed, dissolved in 30.0 mL of DMF then activated by combining with an HBTU solution in DMF (concentration: 205.76 g/L; 21.4 mL; 2.0 equiv. based on Fmoc-Glu(OtBu)-O-2CT Resin) and then adding DIEA (4.5 mL; 4.5 equiv. based on Fmoc-Glu(OtBu)-O-2CT Resin) at 0°-5° C. The resulting solution was added to reaction vessel containing resin, the activation flask was rinsed with 18.7 mL DCM into reactor, which was then stirred at 25° C. After 6.0 hours stirring coupling reaction mixture, the coupling solution was drained and the resin was washed with DMF 4 times (120 mL).

All reagents used in this example are listed in following table:

Coupling Reaction of the GPA Fmoc-AA(19-27)-O-2CT Example 2

| Amino | | HOBT | DIEA | DMF | HBTU | DMF | DCM | Coupling |
|---|---|---|---|---|---|---|---|---|
| Acid | g | H₂O (g) | (mL) | (mL) | (g) | (mL) | (mL) | time (min) |
| Ser(OtBu) | 4.48 | 1.78 | 4.5 | 30.0 | 4.4 | 17.0 | 18.7 | 360 |

The built resin was sequentially washed with DMF (120 mL.) 4 times, DCM (120 mL.) 8 times and IPA (120 mL) 4 times.

### Example 8

Cleavage of the GLP-1 Fragment Fmoc-AA(18-27)-OH from Built Resin

The built resin was swelled with the DCM (200 mL) for 30 min. and then cooled to −5° C. The DCM was drained and the cold solution of 1% v/v TFA/DCM (200 mL at −5° to −10° C.) was added and stirred at 0° C. Pyridine (6.5 mL) was added to the cleavage receiver for the neutralization of the TFA from the cleavage solution. After 30 min stirring, the cleavage solution was collected in the cleavage receiver. Then another cold solution of 1% TFA/DCM (200 mL at −5° to −10° C.) was added and stirred for 30 min at 0° C. After drain the second cleavage solution into cleavage receiver, IPA (20 mL) was charged to cleavage receiver to avoid the gel formation. Cleavage vessel was warmed up to 25° C., and the resin was washed with DCM (200 mL) 6 times and drained into the cleavage solution receiver. The combining cleavage and wash solution were concentrated to a volume of less than 200 mL and then combined with water (200 mL). The DCM was distilled under reduced pressure with vigorously agitation (350-50 torr at 25° C.). The fragment precipitated out from the water mixture when the DCM was removed. The fragment was filtered, washed with water, and dried at 30°-35° C. under vacuum. A total of 18.09 g of GLP-1 Fragment Fmoc-AA(18-27)-OH with a purity of 89.0% AN was obtained, yield of 82.7%.

### Solid Phase Synthesis of GLP-1 Fragment 3, Fmoc-AA(28-35)-OH

Fmoc-Phe-Ile-Ala-Trp(Boc)-Leu-Val-Lys(Boc)-Aib-OH

### Example 9

### Solid Phase Synthesis of the GLP-1 Alternative Fragment 3, Fmoc-AA(28-35)-O-2CT

Fmoc-Phe-Ile-Ala-Trp(Boc)-Leu-Val-Lys(Boc)-Aib-2-
CTC

Solid phase synthesis of Fmoc-AA(28-35)-O-2CT was carried out on Roche Peptide Synthesizer. 15.02 g of Fmoc-Aib-2-CTC resin with loading factor at 0.36 mmoles/g were charged to reaction vessel and swelled in DCM (150 mL) for 30 min at 25° C. The DCM solvent was drained and the resin was washed three times with DMF (6 vol. each wash). All deprotections of resin were carried out by treating the resin twice with 20% piperidine in DMF (6 vol. each treatment) to remove Fmoc protecting groups. After the second 20% piperidine/DMF treatment, the resin was washed nine times with DMF (6.7 vol. each wash).

To prepare the coupling solution, the amino acid and 1-Hydroxybenzotriazole Hydrate (HOBT.H₂O) were weighed, dissolved in DMF then sequentially combined with HBTU solution (0.503 mmoles/mL)in DMF and DIEA at 0°-5° C. The resultant solution was added to reaction vessel, flask was rinsed with DCM into reactor, which was stirred with resin for 4-16 hours at 25° C. The sample was pulled for Kaiser Test or HPLC analysis to check the reaction completion. After the coupling reaction was completed, the coupling solution was drained and the resin was washed with NMP 4 times (6.7 vol. each wash). Then the deprotecting of the Fmoc group and coupling reaction cycle was repeated for remaining amino acid in the fragment (i.e., in the order of Lys(Boc)→Val→Leu→Trp(Boc)→Ala→Ile→Phe.

Due to a buttressing effect between 2-methylalanine (Aib) and 2-CTC resin there is considerable difficulty to force the first two amino acid coupling reactions (Lys(Boc)-34 and Val-33) to completion. The Coupling conditions for Lys(Boc)-34 and Val-33 were modified by increasing the usages of both amino acid and HOBT Hydrate from 1.7 equiv. to 2.35 equiv. and DIEA from 4.0 equiv. to 5.0 equiv. to force the coupling reaction to completion. Also, in this example, acetic anhydride was used to end-capping any unreacting peptide fragment or amino acid on the resin after coupling reactions of Lys(Boc)-34 and Val-33. This has improved the efficiency of the purification step by moving the impurities far from the desirable product during chromatographic purification.

All reagents used in this example are listed in following table:

| | A.A wt | HOBT.H2O | DMF | DIEA | HBTU Sol’n | DCM | Coupling |
|---|---|---|---|---|---|---|---|
| Material | (g)/Eq | (g/Eq) | (mL) | (mL/Eq) | (mL/Eq) | (mL) | time (min) |
| Lys(Boc) | 5.97/2.35 | 1.94/2.35 | 36.8 | 4.7/5.0 | 25.2/2.35 | 21.4 | 960 |
| Acetic | 2.81/5.0 | — | — | 9.4/10.0 | — | — | 180 |
| Anhydride | | | | | | | |
| Val | 4.31/2.35 | 1.97/2.35 | 36.8 | 4.7/5.0 | 25.2/2.35 | 21.4 | 960 |
| Acetic | 2.79/5.0 | — | — | 9.4/10.0 | — | — | 180 |
| Anhydride | | | | | | | |
| Leu | 3.26/1.7 | 1.42/1.7 | — | 3.8/4.0 | 18.2/1.7 | 21.4 | 240 |
| Trp(Boc) | 4.83/1.7 | 1.42/1.7 | — | 3.8/4.0 | 18.2/1.7 | 21.4 | 240 |
| Ala | 3.05/1.7 | 1.42/1.7 | — | 3.8/4.0 | 18.2/1.7 | 21.4 | 240 |
| Ile | 3.27/1.7 | 1.43/1.7 | — | 3.8/4.0 | 18.2/1.7 | 21.4 | 240 |
| Phe | 3.57/1.7 | 1.43/1.7 | — | 3.8/4.0 | 18.2/1.7 | 21.4 | 240 |

After completion of the solid phase synthesis the resin were washed by DMF (4×6.7 vol), DCM (7×6.7), and isopropanol (3×6.7 vol). The vacuum dried the built resin and hold for cleavage.

### Example 10

Cleavage of the GLP-1 Intermediate Fragment Fmoc-AA(28-35)-OH from Built Resin

The built resin from above was swelled in DCM (10 vol) for 30 min at 25° C. Then cooled the pot mixture to −5° C. The DCM was drained and the resin was treated with the cold solution of 2% TFA/DCM (2×7.5 vol) twice by stirring for 30 min at 0° C. The cleavage solution was collected in the flask containing pyridine (1.3 equiv. of the total of TFA). While vessel warming up to 25° C., the resin was washed with DCM 6 times (10 vol.) and drained into the DCM washes. The DCM solution was combined, concentrated, and mixed with water (10 vol.). The resultant mixture was distilled under reduced pressure to remove DCM (350-50 torr at 25° C.). The fragment precipitated out from water when DCM was removed. The fragment was filtered, washed with and dried at 30°-35° C. under vacuum. A 92.7% yield of GLP-1 Fmoc-AA(28-35)-OH was obtained with a purity of 95.2% AN.

### Example 11

### Solid Phase Synthesis of the GLP-1 Fragment 3 Fmoc-AA(28-35)-O-2CT

Fmoc-Phe-Ile-Ala-Trp(Boc)-Leu-Val-Lys(Boc)-Aib-2-
CTC

Solid phase synthesis of Fmoc-AA(28-35)-O-2CT was carried out on Roche Peptide Synthesizer. 25.01 g of H-Aib-2-CTC resin with loading factor at 0.59 mmoles/g (batch #BO06010051) were charged to reaction vessel and swelled in DCM (250 mL) for 30 min at 25° C. The DCM solvent was drained and the resin was washed three times with NMP (6 vol. each wash).

All deprotections of resin were carried out by treating the resin twice with 20% piperidine in NMP (5.6 vol. each treatment) to remove Fmoc protecting groups. After the second 20% piperidine/NMP treatment, the resin was washed nine times with NMP (5.6 vol. each wash).

To prepare the coupling solution, the amino acid and 1-Hydroxybenzotriazole Hydrate (HOBT.H2O) were weighed, dissolved in NMP then sequentially combined with HBTU solution (0.46 mmoles/mL)in NMP and DIEA at 0°-5° C. The resultant solution was added to reaction vessel, flask was rinsed with NMP into reactor, which was stirred with resin for 4-16 hours at 25° C. The sample was pulled for Kaiser Test or HPLC analysis to check the reaction completion. After the coupling reaction was completed, the coupling solution was drained and the resin was washed with NMP 4 times (6.7 vol. each wash). Then the deprotecting of the Fmoc group and coupling reaction cycle was repeated for remaining amino acid in the fragment (i.e., in the order of Lys(Boc)→Val→Leu→Trp(Boc)→Ala→Ile→Phe).

The Coupling conditions for Lys(Boc)-34 and Val-33 were modified by increasing the usages of both amino acid and HOBT Hydrate from 1.7 Eq to 2.0 Eq and DIEA from 2.13 Eq to 2.5 Eq to force the coupling reaction to completion. Also, in this example, acetic anhydride in DCM was used to end-capping any unreacting peptide fragment or amino acid on the resin after coupling reactions of Lys(Boc)-34 and Val-33.

All reagents used in this example are listed in following table:

| | A.A wt | HOBT.H2O | NMP | DCM | DIEA | HBTU Sol’n | NMP Resin | DCM Rinse | Coupling |
|---|---|---|---|---|---|---|---|---|---|
| Material | (g)/Eq | (g/Eq) | (mL) | (mL) | (mL/Eq) | (mL/Eq) | (mL) | (mL) | time (min) |
| Lys(Boc) | 13.86/2.0 | 0.24/0.1 | 50.5 | — | 6.4/2.5 | 64.2/2.0 | 37.5 | — | 720 |
| Acetic | 4.62/3.0 | — | — | 75.0 | 12.8/5.0 | — | — | 37.5 | 120 |
| Anhydride | | | | | | | | | |
| Val | 4.31/2.35 | 0.26/0.1 | 50.5 | — | 6.4/2.5 | 64.2/2.0 | 37.5 | — | 720 |
| Acetic | 4.60/3.0 | — | — | 75.0 | 12.8/5.0 | — | — | 37.5 | 120 |
| Anhydride | | | | | | | | | |
| Leu | 8.85/1.7 | 0.22/0.085 | 58.4 | — | 5.5/2.13 | 54.6/1.7 | 37.5 | — | 240 |
| Trp(Boc) | 13.22/1.7 | 0.21/0.085 | 58.4 | — | 5.5/2.13 | 54.6/1.7 | 37.5 | — | 240 |
| Ala | 8.28/1.7 | 0.21/0.085 | 58.4 | — | 5.5/2.13 | 54.6/1.7 | 37.5 | — | 240 |
| Ile | 8.85/1.7 | 0.19/0.085 | 58.4 | — | 5.5/2.13 | 54.6/1.7 | 37.5 | — | 240 |
| Phe | 9.73/1.7 | 0.21/0.085 | 58.4 | — | 5.5/2.13 | 54.6/1.7 | 37.5 | — | 240 |

After completion of the solid phase synthesis the resin were washed by NMP (4×6.0 vol), DCM (7×6.0 vol).

Cleavage of the GLP-1 Intermediate Fragment Fmoc-AA(28-35)-OH from Built Resin

The built resin from above was cooled in DCM (6 vol) for 30 min to −5° C. Then DCM was drained and the resin was treated with the cold solution of 2% TFA/DCM (2×10 vol) twice by stirring for 30 min at 0° C. The cleavage solution was collected in the flask containing pyridine (1.3 equiv. of the total of TFA). While vessel warming up to 25° C., the resin was washed with DCM 6 times (10 vol.) and drained into the DCM washes. The DCM solution was combined, concentrated, and mixed with water (6 vol.). The resultant mixture was distilled under reduced pressure to remove DCM (350-50 torr at 25° C.). The fragment precipitated out from water when DCM was removed. The fragment was filtered, washed with and dried at 30°-35° C. under vacuum. A 96.9% yield of Fmoc-AA(28-35)-OH was obtained with a purity of 96.1% AN.

### The Solution Phase Synthesis of the GLP-1 Fragment 3′, H-AA(28-36)-NH2

H-Phe-Ile-Ala-Trp(Boc)-Leu-Val-Lys(Boc)-Aib-Arg-
NH₂

### Example 12

The alternative fragment 3 (Fmoc-AA(28-35)-OH, 6.11 g, 4.42 mmoles, and Argininamide Dihydrochloride (H-Arg (2HCl)-NH2, 2.18 g, 8.84 mmoles, 2 equiv, Lot #BO06110014) were dissolved in DMF (42 mL). To this solution, the solution of HOBt.H2O (0.67 g, 1 equiv) and HBTU (3.38 g, 2 equiv) in DMF (42 mL), DIEA (3.44 mL, 4 equiv) were sequentially charged along with 15 mL of DMF. The reaction was agitated at 25° C. and monitored by HPLC. After 2 hours, the reaction was not completed. The reaction was done overnight (21 hours). Then piperidine (2.26 g, 6 equiv) was added to the reaction mixture. The Fmoc removal was not completed after stirring at 35° C. for one hour. The additional piperidine (2.33 g, 6.2 equiv) was added and stirring another 1.75 hours. The reaction mixture was quenched with water (240 mL). Pyridine hydrochloride (8.33 g, 16.3 equiv) was charged to the precipitated pot mixture to neutralize the piperidine. The white solid formed was filtered, washed with water (400 mL) and partially dried overnight. After the filter cake was reslurried with 100 mL MTBE/n-heptane (1:1=vol: vol), filtered, washed with MTBE/n-heptane (1:1=vol:vol; 2×25 mL), and vacuum dried to give GLP-1 alternative Fragment 3′ H-AA(28-36)-NH2 (6.22 g, weight yield 106.9%). HPLC analysis shown a purity of 87% AN.

### Example 13

The alternative fragment 3 (Fmoc-AA(28-35)-OH, 6.12 g, 4.42 mmoles and Argininamide Dihydrochloride (H-Arg (2HCl)-NH2, 2.19 g, 8.84 mmoles, 2 equiv, Lot #BO06110014) were dissolved in DMF (42 mL). To this solution, the solution of HOBt.H2O (0.67 g, 1 equiv) and HBTU (3.38 g, 2 equiv) in DMF (42 mL), DIEA (3.44 mL, 4 equiv) were sequentially charged along with 15 mL of DMF. The reaction was agitated at 25° C. and monitored by HPLC. The reaction was done overnight (16.3 hours). Then piperidine (4.52 g, 12 equiv) was added to the reaction mixture. The Fmoc removal was completed after stirring at 25° C. for 35 min. The reaction mixture was quenched with water (200 mL). 180 mL DCM were charged and extracted the precipitated product. The bottom DCM layer was washed with water twice (2×100 mL) and concentrated to a volume of 50 mL. This concentrated DCM solution was feed by portion to precipitate the product. DCM was distilled by vacuum. MTBE was charged to the precipitation mixture. The white solid formed was filtered, washed with MTBE/n-heptane (1:1=vol: vol; 2×50 mL), and vacuum dried to give the GLP-1 alternative Fragment 3′ H-AA(28-36)-NH2(6.54 g, weight yield 112.4%). HPLC analysis shown a purity of 92.1% AN.

### The Solution Phase Synthesis of the Crude GLP-1 Following Scheme 2

### Example 14

The GLP-1 Fragment Fmoc-AA(18-27)-OH (1.87 g) was mixed with 2-Me-THF (20 mL) and DMSO (5 mL) at 22° C. Then this solution was combined with Fragment H-AA(28-36)-NH2) (1.30 g, 1.0 equiv.) and stirred. To this cloudy suspension, DEPBT (0.41 g, 1.3 equiv.), and then DIEA (0.40 mL, 2.3 equiv) were charged along with a Me-THF rinse (5 mL). The reaction was agitated at room temperature (22° C.) and monitored by HPLC. After 4.5 h a reaction completion check indicated that the reaction was incomplete (16.8% excess of Fragment Fmoc-AA(18-27)-OH). Kicker charges of Fragment H-AA(28-36)-NH2) (0.43 g), DEPBT (0.13 g), and DIEA (0.08 mL) were added. After stirring overnight at room temperature, HPLC analysis indicated that the reaction was complete. Piperidine (0.30 mL, 3 equiv.) was charged and the resulting reaction mixture was stirred at room temperature. After stirring for 1 h, the de-Fmoc completion check indicated that the reaction was incomplete. A kicker charge of Piperidine (0.30 mL) was added. A sample taken after an overnight stir indicated that the de-protection reaction was done. Water (35 mL) was charged to quench the reaction and extract the organic phase. After separation of phases, Me-THF (20 mL) was added to the aqueous phase as a back-extraction. The combined organic phases were distilled to an oil under vacuum (95 torr, bath 37° C.), re-dissolved in Me-THF (30 mL) and again distilled to an oil. The oil was again dissolved in Me-THF (30 mL) and the resulting mixture (along with a Me-THF (10 mL) rinse) was poured into a reaction vessel at 15° C. containing n-heptane (60 mL). After 30 min of aging, the precipitated product was filtered, washed with n-heptane (20 mL) and dried overnight to afford 2.78 g of product with the purity of 55.2% AN, 94.1% yield based on Fragment Fmoc-AA(18-27)-OH.

### Example 15

The GLP-1 Fragment Fmoc-AA(7-17)-OH (1.00 g) was dissolved in THF (20 mL) at 22° C. Then this solution was combined with Fragment H-AA(18-36)-NH2) (1.81 g, 1.2 equiv.) and stirred to dissolve all solids. To this solution, DEPBT (0.181 g, 1.2 equiv.), and then DIEA (0.22 mL, 2.4 equiv) were charged along with a THF rinse (5 mL). The reaction was agitated at room temperature (22° C.) and monitored by HPLC. After 2.5 h, reaction completion check indicated that the reaction was incomplete (21.9% excess of Fragment Fmoc-AA(7-17)-OH). Kicker charges of Fragment H-AA(18-36)-NH2) (0.80 g), DEPBT (0.10 g), and DIEA (0.11 mL) were added. After stirring overnight at room temperature, HPLC analysis indicated that the reaction was complete. Piperidine (0.30 mL, 6 equiv.) was charged and the resulting reaction mixture was stirred at room temperature. After stirring for 5 h, the de-protection reaction was done. The THF in the reaction mixture then was displaced with DCM (13 mL) under vacuum (35° C. under 50 mm Hg vacuum). The residue was dissolved in DCM (10 mL) and combined with a solution of DTT (2.04 g), water (2.0 g), and TFA (40 mL) with a DCM rinse (2 mL) at 15° C. After stirring 6 hours at 15° C., the reaction mixture was cooled to −3° C. and quenched by charging cold (−20° C.) MTBE (180 mL). The quenched reaction mixture was aged at 15° for 30 min. The solid product was filtered, washed with MTBE (3×50 mL), and dried overnight. A 2.78 g of GPA crude (21.0% wt/wt) was obtained with a purity of 38.9% AN; 163.6% yield (based on Fragment Fmoc-AA(7-17)-OH).

The features disclosed in the foregoing description, or the following claims, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

What is claimed is:

1. A method of making an insulinotropic peptide, comprising the steps of:
a) providing a first peptide fragment including the amino acid sequence of (SEQ ID NO. 5)
Z-QAAKEFIAWLVKX³⁵R-NH₂
wherein
Z is H—;
X³⁵ is an achiral, optionally sterically hindered amino acid residue; and
one or more residues of the sequence optionally includes side chain protection;
b) providing a second peptide fragment including the amino acid sequence of (SEQ ID NO. 6)
Z-SYLEG
wherein
Z is an N-terminal protecting group; and
one or more residues of the sequence optionally includes side chain protection;
c) coupling the first peptide fragment to the second peptide fragment in solution in order to provide a third peptide fragment including the amino acid sequence of (SEQ ID NO. 7)
Z-SYLEGQAAKEFIAWLVKX³⁵R-NH₂
wherein
Z is an N-terminal protecting group;
X³⁵ is an achiral, optionally sterically hindered amino acid residue; and
one or more residues of the sequence optionally includes side chain protection;
d) removing the N-terminal protecting group of the third peptide fragment to afford a fourth peptide fragment including the amino acid sequence of (SEQ ID NO. 7)
Z-SYLEGQAAKEFIAWLVKX³⁵R-NH₂
wherein
Z is H—;
X³⁵ is an achiral, optionally sterically hindered amino acid residue; and
one or more residues of the sequence optionally includes side chain protection;
e) providing a fifth peptide fragment including the amino acid sequence of (SEQ ID NO. 8)
Z-HX⁸EGTFTSDVS-B′
wherein
X⁸ is an achiral, optionally sterically hindered amino acid residues;
Z is an N-terminal protecting group;
B′ is —OH; and
one or more residues of the sequence optionally includes side chain protection;
f) coupling the fifth peptide fragment to the fourth peptide fragment in solution to provide an insulinotropic peptide including the amino acid sequence of (SEQ ID NO. 9)
Z-HX⁸EGTFTSDVSSYLEGQAAKEFIAWLVKX³⁵R-NH₂
wherein
Z is an N-terminal protecting group;
X⁸ and X³⁵ are each independently achiral, optionally sterically hindered amino acid residues; and
one or more residues of the sequence optionally includes side chain protection.

2. The method of claim 1, further comprising the steps of:
g) removing the N-terminal protecting group of the insulinotropic peptide resulting from step f) to afford the insulinotropic peptide including amino acid sequence of (SEQ ID NO. 9)
Z-HX⁸EX¹⁰TFTSDVSSYLEGQAAKEFIAWLVKX³⁵R-NH₂
wherein
Z is H—;
X⁸ and X³⁵ are each independently achiral, optionally sterically hindered amino acid residues; and
one or more residues of the sequence optionally includes side chain protection; and
h) contacting the insulinotropic peptide resulting from step g) with acid in order to deprotect the amino acid side chains to afford the deprotected insulinotropic peptide including amino acid sequence of (SEQ ID NO. 9)
Z-HX⁸EGTFTSDVSSYLEGQAAKEFIAWLVKX³⁵R-NH₂
wherein
Z is H—; and
X⁸ and X³⁵ are each independently achiral, optionally sterically hindered amino acid residues.

3. The method of claim 2, wherein the deprotected insulinotropic peptide resulting from step h) has the amino acid sequence (SEQ. ID No. 4)
HAibEGTFTSDVSSYLEGQAAKEFIAWLVKAibR

4. A method of making an insulinotropic peptide, comprising the steps of:
a) providing a first peptide fragment including the amino acid sequence of (SEQ ID NO. 8)
Z-HX⁸EGTFTSDVS-B′
wherein
X⁸ is an achiral, optionally sterically hindered amino acid residues;
Z is an N-terminal protecting group;
B′ is —OH; and
one or more residues of the sequence optionally includes side chain protection;
b) providing a second peptide fragment including the amino acid sequence of (SEQ ID NO. 6)
Z-SYLEG-B′
wherein
B′ is a solid phase resin;
Z is H—; and
one or more residues of the sequence optionally includes side chain protection;
c) coupling the first peptide fragment to the second peptide fragment in order to provide a third peptide fragment including the amino acid sequence of (SEQ ID NO. 11)
Z-HX⁸EGTFTSDVSSYLEG-B′
wherein
B′ is a solid phase resin;
Z is an N-terminal protecting group; and
one or more residues of the sequence optionally includes side chain protection;
d) removing the third peptide fragment from the solid phase resin to provide a fourth peptide fragment including the amino acid sequence of (SEQ ID NO. 11)
Z-HX⁸EGTFTSDVSSYLEG-B′
wherein
B′ is —OH;
Z is an N-terminal protecting group; and
one or more residues of the sequence optionally includes side chain protection;
e) providing a fifth peptide fragment including the amino acid sequence of (SEQ ID NO. 5)
Z-QAAKEFIAWLVKX³⁵R-NH₂
wherein
Z is H—;
X³⁵ is an achiral, optionally sterically hindered amino acid residue; and
one or more residues of the sequence optionally includes side chain protection;
f) coupling the fourth peptide fragment to the fifth peptide fragment in solution to provide an insulinotropic peptide including the amino acid sequence of (SEQ ID NO. 9)
Z-HX⁸EGTFTSDVSSYLEGQAAKEFIAWLVKX³⁵R-NH₂
wherein
Z is an N-terminal protecting group;
X⁸ and X³⁵ are each independently achiral, optionally sterically hindered amino acid residues; and
one or more residues of the sequence optionally includes side chain protection.

5. The method of claim 4, further comprising the steps of:
g) removing the N-terminal protecting group of the insulinotropic peptide resulting from step f) to afford an insulinotropic peptide including the amino acid sequence of (SEQ ID NO. 7)
Z-HX⁸EGTFTSDVSSYLEGQAAKEFIAWLVKX³⁵R-NH₂
wherein
Z is H—;
X⁸ and X³⁵ are each independently achiral, optionally sterically hindered amino acid residues; and
one or more residues of the sequence optionally includes side chain protection;
h) contacting the insulinotropic peptide resulting from step g) with acid in order to deprotect the amino acid side chains to afford the deprotected insulinotropic peptide including amino acid sequence of (SEQ ID NO. 9)
Z-HX⁸EGTFTSDVSSYLEGQAAKEFIAWLVKX³⁵R-NH₂
wherein
Z is H—; and
X⁸ and X³⁵ are each independently achiral, optionally sterically hindered amino acid residues.

6. The method of claim 5, wherein the deprotected insulinotropic peptide has the amino acid sequence (SEQ. ID No. 4)
HAibEGTFTSDVSSYLEGQAAKEFIAWLVKAibR-NH₂

7. A method of making an insulinotropic peptide, comprising the steps of:
a) providing a first peptide fragment including the amino acid sequence of (SEQ ID NO. 12)
Z-SYLEGQAAKE-B′
wherein
Z is H—; and
B′ is a solid phase resin;
b) providing a second peptide fragment including the amino acid sequence of (SEQ ID NO. 8)
Z-HX⁸EGTFTSDVS-B′
wherein
X⁸ is an achiral, optionally sterically hindered amino acid residues;
Z is an N-terminal protecting group;
B′ is —OH; and
one or more residues of the sequence optionally includes side chain protection;
c) coupling the second peptide fragment to the first peptide fragment to provide a third peptide fragment including the amino acid sequence of (SEQ ID NO. 13)
Z-HX⁸EGTFTSDVSSYLEGQAAKE-B′
wherein
Z is an N-terminal protecting group;
B′ is a solid phase resin;
X⁸ is an achiral, optionally sterically hindered amino acid residues; and
one or more residues of the sequence optionally includes side chain protection.

8. The method of claim 7, further comprising the steps of:
d) removing the third peptide fragment from the solid phase resin to provide a fourth peptide fragment including amino acid sequence of (SEQ ID NO. 13)
Z-HX⁸EGTFTSDVSSYLEGQAAKE-B′
wherein
Z is H—;
B′ is —OH;
X⁸ is an achiral, optionally sterically hindered amino acid residues; and
one or more residues of the sequence optionally includes side chain protection; and
e) providing a fifth peptide fragment including the amino acid sequence of (SEQ ID NO. 14)
Z-FIAWLVKX³⁵R-NH₂
wherein
X³⁵ is an achiral, optionally sterically hindered amino acid residue; and
one or more residues of the sequence optionally includes side chain protection;
f) coupling the fourth peptide fragment to the fifth peptide fragment in solution to provide an insulinotropic peptide including the amino acid sequence of (SEQ ID NO. 9)
Z-HX⁸EGTFTSDVSSYLEGQAAKEFIAWLVKX³⁵R-NH₂
wherein
Z is an N-terminal protecting group;
X⁸ and X³⁵ are each independently achiral, optionally sterically hindered amino acid residues; and
one or more residues of the sequence optionally includes side chain protection;
g) removing the N-terminal protecting group of the insulinotropic peptide resulting from step f) to afford the insulinotropic peptide including amino acid sequence of (SEQ ID NO. 9)
Z-HX⁸EGTFTSDVSSYLEGQAAKEFIAWLVKX³⁵R-NH₂
wherein
Z is H—;
X⁸ and X³⁵ are each independently achiral, optionally sterically hindered amino acid residues; and
one or more residues of the sequence optionally includes side chain protection; and
h) contacting the insulinotropic peptide resulting from step g) with acid in order to deprotect the amino acid side chains to afford the deprotected insulinotropic peptide including amino acid sequence of (SEQ ID NO. 9)
Z-HX⁸EGTFTSDVSSYLEGQAAKEFIAWLVKX³⁵R-NH₂
wherein
Z is H—; and
X⁸ and X³⁵ are each independently achiral, optionally sterically hindered amino acid residues.

9. The method of claim 8, wherein the deprotected insulinotropic peptide has the amino acid sequence (SEQ. ID No. 4)
HAibEGTFTSDVSSYLEGQAAKEFIAWLVKAibR-NH₂

10. A method of making an insulinotropic peptide, comprising the steps of:
a) providing a first peptide fragment including the amino acid sequence of (SEQ ID NO 14)
Z-FIAWLVKX³⁵R-NH₂
wherein
Z is H—;
X³⁵ is an achiral, optionally sterically hindered amino acid residue; and
one or more residues of the sequence optionally includes side chain protection;
b) providing a second peptide fragment including the amino acid sequence of (SEQ ID NO. 12)
Z-SYLEGQAAKE-B′
wherein
Z is an N-terminal protecting group;
B′ is —OH; and
one or more residues of the sequence optionally includes side chain protection;
c) coupling the first peptide fragment to the second peptide fragment in solution to provide a third peptide fragment including the amino acid sequence of (SEQ. ID NO. 7)
Z-SYLEGQAAKEFIAWLVKX³⁵R-NH₂
wherein
Z is an N-terminal protecting group;
X³⁵ is an achiral, optionally sterically hindered amino acid residues; and
one or more residues of the sequence optionally includes side chain protection;

11. The method of claim 10, further comprising the steps of:
d) removing the N-terminal protecting group of the third peptide fragment to afford a fourth peptide fragment including the amino acid sequence of (SEQ. ID NO. 7)
Z-SYLEGQAAKEFIAWLVKX³⁵R-NH₂
wherein
Z is H—;
X³⁵ is an achiral, optionally sterically hindered amino acid residues; and
one or more residues of the sequence optionally includes side chain protection;
e) providing a fifth peptide fragment including the amino acid sequence of (SEQ ID NO. 8)
Z-HX⁸EGTFTSDVS-B′
wherein
X⁸ is an achiral, optionally sterically hindered amino acid residues;
Z is an N-terminal protecting group;
B′ is —OH; and
one or more residues of the sequence optionally includes side chain protection;
f) coupling the fifth peptide fragment to the fourth peptide fragment in solution to provide an insulinotropic peptide including the amino acid sequence of (SEQ ID NO. 9)
Z-HX⁸EGTFTSDVSSYLEGQAAKEFIAWLVKX³⁵R-NH₂
wherein
Z is H—; and
X⁸ and X³⁵ are each independently achiral, optionally sterically hindered amino acid residues;
g) removing the N-terminal protecting group of the insulinotropic peptide resulting from step f) to afford the insulinotropic peptide including amino acid sequence of (SEQ ID NO. 9)
Z-HX⁸EGTFTSDVSSYLEGQAAKEFIAWLVKX³⁵R-NH₂
wherein
Z is H—;
X⁸ and X³⁵ are each independently achiral, optionally sterically hindered amino acid residues; and
one or more residues of the sequence optionally includes side chain protection; and
h) contacting the insulinotropic peptide resulting from step h) with acid in order to deprotect the amino acid side chains to afford the deprotected insulinotropic peptide including amino acid sequence of (SEQ ID NO. 9)
Z-HX⁸EGTFTSDVSSYLEGQAAKEFIAWLVKX³⁵R-NH₂
wherein
Z is H—; and
X⁸ and X³⁵ are each independently achiral, optionally sterically hindered amino acid residues.

12. The method of claim 11, wherein the deprotected insulinotropic peptide has the amino acid sequence (SEQ. ID No. 4)
HAibEGTFTSDVSSYLEGQAAKEFIAWLVKAibR-NH₂

13. A peptide of the amino acid sequence (SEQ ID NO. 5)
Z-QAAKEFIAWLVKX³⁵R-NH₂
wherein
Z is H— or an N-terminal protecting group;
X³⁵ is an achiral, optionally sterically hindered amino acid residue; and
one or more residues of the sequence optionally includes side chain protection.

14. A peptide of the amino acid sequence (SEQ ID NO. 7)
Z-SYLEGQAAKEFIAWLVKX³⁵R-NH₂
wherein
Z is H— or an N-terminal protecting group;
X³⁵ is an achiral, optionally sterically hindered amino acid residue; and
one or more residues of the sequence optionally includes side chain protection.

15. A peptide of the amino acid sequence (SEQ ID NO. 8)
Z-HX⁸EGTFTSDVS-B′
wherein
X⁸ is an achiral, optionally sterically hindered amino acid residues;
Z is H— or an N-terminal protecting group;
B′ is —OH or a solid phase resin; and
one or more residues of the sequence optionally includes side chain protection.

16. A peptide of the amino acid sequence (SEQ ID NO. 11)
Z-HX⁸EGTFTSDVSSYLEG-B′
wherein
B′ is —OH or a solid phase resin;
Z is H— or an N-terminal protecting group; and
one or more residues of the sequence optionally includes side chain protection.

17. A peptide of the amino acid sequence (SEQ ID NO. 12)
Z-SYLEGQAAKE-B′
wherein
Z is H— or an N-terminal protecting group; and
B′ is —OH or a solid phase resin.

18. A peptide of the amino acid sequence (SEQ ID NO. 13)
Z-HX⁸EGTFTSDVSSYLEGQAAKE-B′
wherein
Z is H— or an N-terminal protecting group;
B′ is —OH or a solid phase resin;
X⁸ is an achiral, optionally sterically hindered amino acid residues; and
one or more residues of the sequence optionally includes side chain protection.

19. A peptide of the amino acid sequence (SEQ. ID NO. 7)
Z-SYLEGQAAKEFIAWLVKX³⁵R-NH₂
wherein
Z is H— or an N-terminal protecting group;
X³⁵ is an achiral, optionally sterically hindered amino acid residues; and
one or more residues of the sequence optionally includes side chain protection.

20. A peptide of the amino acid sequence (SEQ. ID NO. 14)
Z-FIAWLVKX³⁵R-NH₂
wherein
Z is H— or an N-terminal protecting group;
X³⁵ is an achiral, optionally sterically hindered amino acid residues; and
one or more residues of the sequence optionally includes side chain protection.

21. The peptide of any one of claims 13 to 20 wherein Z is Fmoc.
